(19) United States
(12) Patent Application Publication
(10) Pub. No.: US 2014/0186376 A1
(43) Pub. Date: Jul. 3, 2014

(54) Compositions and Methods for Inhibiting CCL3

(71) Applicant: University of Rochester, Rochester, NY (US)

(72) Inventors: Benjamin J. Frisch, Rochester, NY (US); Laura M. Calvi, Rochester, NY (US); Michael W. Becker, Pittsford, NY (US)

(73) Assignee: University of Rochester, Rochester, NY (US)

(21) Appl. No.: 14/194,344

(22) Filed: Feb. 28, 2014

Related U.S. Application Data
(63) Continuation-in-part of application No. PCT/US12/52854, filed on Aug. 29, 2012.

(51) Int. Cl. C07K 16/18; C07K 14/52; C07K 14/435

(52) U.S. Cl. CPC C07K 16/18; C07K 14/435; C07K 14/522
USPC 424/174.1; 514/19.3; 514/44 A

(57) ABSTRACT

The present invention relates to the discovery that CCL3, through at least one of its receptors CCR1 and CCR5, plays a role in the pathologies associated with myeloid neoplasms. In various embodiments, the pathologies associated with myeloid neoplasms treatable by the compositions and methods of the invention described herein include, but are not limited to, at least one of osteoclast/osteoblast imbalance, inhibition of osteoblast function, bone loss, disregulation of the hematopoietic stem cell microenvironment, abnormal hematopoietic recovery, pancytopenia, anemia, thrombocytopenia, neutropenia, bicytopenia, and erythrocytopenia. Interfering with the interaction between CCL3 and its receptors, by targeting at least one of CCL3, CCR1, CCR5, diminishes the effects of pathologies associated with myeloid neoplasms. In various embodiments, the myeloid neoplasm is a myeloproliferative disorder, acute myeloid leukemia (AML), chronic myeloid leukemia (CML), chronic myelomonocytic leukemia (CMML), or myelodysplastic syndrome (MDS).

### CROSS REFERENCE TO RELATED APPLICATIONS

This application is a continuation-in-part of PCT Application No. PCT/US12/52854, filed Aug. 29, 2012, which claims the benefit of priority to U.S. Provisional Patent Application No. 61/528,410, filed on Aug. 29, 2011, the contents of each of which are incorporated by reference herein in their entireties.

### BACKGROUND OF THE INVENTION

Efficacy of treatment for acute myeloid leukemia (AML), the most common adult acute leukemia, is limited and recurrence is common. One of the major causes of morbidity and mortality of acute leukemia is the disruption of normal hematopoiesis, causing neutropenia, anemia, and thrombocytopenia. Hematopoietic damage often occurs prior to overt systemic leukemia, which suggests that leukemic cells play an active role in the inhibition of normal hematopoiesis. The mechanisms by which AML inhibits normal hematopoiesis are poorly understood and it is unclear whether this is a direct effect of the leukemic cells on the normal hematopoietic cells in the marrow, or whether the microenvironment mediates leukemia-dependent hematopoietic damage (Lichtman, 2000, Stem Cells 18:304-306).

It has long been known that osteoblastic cells support and expand HSCs in vitro (Taichman et al., 1994, J Exp Med 179:1677-1682), and co-transplantation of osteoblasts with HSCs can increase engraftment rate (El-Badri et al., 1998, Exp Hematol 26:110-116). Previous work first identified osteoblastic cells as a regulatory component in the HSC niche through genetic means (Calvi et al., 2003, Nature 425:841-846; Zhang et al., 2003, Nature 425:836-841). It has become evident that osteoblastic cells can both stimulate (Calvi et al., 2003, Nature 425:841-846; Zhang et al., 2003, Nature 425:836-841) and limit HSC expansion (Nilsson et al., 2005, Blood 106:1232-1239; Stier et al., 2005, J Exp Med 201:1781-1791), promote quiescence (Arai et al., 2004, Cell 118:149-161; Yoshihara et al., 2007, Cell Stem Cell 1:685-697; Fleming et al., 2008, Cell Stem Cell 2:274-283), initiate HSC mobilization (Mayack et al., 2008, Blood 112:519-531), and integrate sympathetic nervous system and HSC regulation (Katayama et al., 2006, Cell 124:407-421). In addition, osteoblastic cells appear to be mediators of the anemia induced by graft vs. host disease after marrow transplantation (Shono et al., 2010, Blood 115:5401-5411), and bone progenitor dysfunction is sufficient to induce myelodysplasia and induce secondary leukemia (Raaijmakers et al., 2010, Nature 464:852-857). Finally, recent data suggest that mesechymal stem cells, which give rise to cells of the osteogenic lineage, regulate HSCs (Mendez-Ferrer et al., 2010, Nature 466:829-834). Therefore, in addition to increasing evidence that osteoblastic lineage cells act as orchestrators of HSC behavior, data strongly suggest that osteoblastic dysfunction results in pancytopenia (Visnjic et al., 2004, Blood 103:3258-3264; Shono et al., 2010, Blood 115:5401-5411). Cells of the mesenchymal/osteoblastic lineage play an essential role in the regulation of normal hematopoietic stem cells (HSCs) (Calvi et al., 2003, Nature 425:841-846; Zhang et al., 2003, Nature 425:836-841; Visnjic et al., 2004, Blood 103:3258-3264). In addition to data suggesting that activation of osteoblastic cells expands HSCs and that osteoblastic injury results in myeloablation (Calvi et al., 2003, Nature 425:841-846; Visnjic et al., 2004, Blood 103:3258-3264), specific disruptions of the osteoblastic compartment without genetic manipulation of the hematopoietic system results in a myeloproliferative disorder, demonstrating the important role osteoblasts play in HSC and progenitor cell regulation (Walkley et al., 2007, Cell 129:1097-1110). Osteoclasts, as well as endothelial cells, have also been shown to play a role in normal hematopoiesis and regulation of HSCs, particularly in their mobilization from the marrow (Kollet et al., 2006, Nat Med 12:657-664; Lymperi et al., 2008, Blood 111:1173-1181; Porter R L et al., 2009, Cell Stem Cell 4:187-188).

In xenograft models, human AML cells reside at the endosteal surface of bone (Ishikawa et al., 2007, Nat Biotechnol 25:1315-1321; Ninomiya et al., 2007, Leukemia 21:136-142), where they are found in close proximity to osteoblastic and osteoclastic cells. However, the interactions between leukemia and these microenvironmental cells have not been clearly defined. Moreover, xenograft models, while beginning to elucidate in vivo intracellular relationships, juxtapose hematopoietic and non-hematopoietic cells from different species, and may not recapitulate normal leukemia-microenvironment regulatory interactions. Nonetheless, a number of xenograft studies have suggested that leukemia disrupts molecular mechanisms used by normal HSCs to home to the endosteal niche, including CD44 (Jin et al., 2006, Nat Med 12:1167-1174; Krause et al., 2006, Nat Med 12:1175-1180) and the well-established interaction between CXCR4 and its ligand CXCL12 (Colmone et al., 2008, Science 322:1861-1865; Rombouts et al., 2004, Blood 104:550-557; Sipkins et al., 2005, Nature 435:969-973).

Thus, there is a need in the art to identify new therapeutic targets for the treatment of myeloid neoplasms and associated pathologies. The present invention addresses this unmet need in the art.

### SUMMARY OF THE INVENTION

The present invention relates to the discovery that CCL3, through at least one of its receptors CCR1 and CCR5, plays a role in pathologies that are associated with myeloid neoplasms. In one embodiment, the invention is a method of treating a pathology associated with myeloid neoplasm in a subject by administering a therapeutically effective amount of a CCL3 inhibitor to the subject, where the subject has myeloid neoplasm, and where after the CCL3 inhibitor is administered to the subject, the pathology is treated. In another embodiment, the invention is a method of preventing a pathology associated with myeloid neoplasm in a subject by administering a therapeutically effective amount of a CCL3 inhibitor to the subject, where the subject has myeloid neoplasm, and where after the CCL3 inhibitor is administered to the subject, the pathology is prevented. In various embodiments, the CCL3 inhibitor useful in the methods of the invention is at least one of a chemical compound, a protein, a peptide, a peptidomemetic, an antibody, a ribozyme, a small molecule chemical compound, or an antisense nucleic acid molecule. In various embodiments, the myeloid neoplasm is at least one of a myeloproliferative disorder, acute myeloid leukemia (AML), chronic myeloid leukemia (CML), chronic myelomonocytic leukemia (CMML) or myelodysplastic syndrome (MDS). In various embodiments, the pathology associated with myeloid neoplasm treatable by the methods of the invention is at least one of osteoclast/osteoblast imbalance, inhibition of osteoblast function, bone loss, disregulation of the hematopoietic stem cell microenvironment, abnormal hematopoietic recovery, pancytopenia, anemia, thrombocytopenia, neutropenia, bicytopenia, or erythrocytopenia. In some embodiments, the subject is a human.

In one embodiment, the invention is a method of treating a pathology associated with myeloid neoplasm in a subject by administering a therapeutically effective amount of a CCL3 receptor inhibitor to the subject, where the subject has myeloid neoplasm, and where after the CCL3 receptor inhibitor is administered to the subject, the pathology is treated. In another embodiment, the invention is a method of preventing a pathology associated with myeloid neoplasm in a subject by administering a therapeutically effective amount of a CCL3 receptor inhibitor to the subject, where the subject has myeloid neoplasm, and where after the CCL3 receptor inhibitor is administered to the subject, the pathology is prevented. In some embodiments, the CCL3 receptor is CCR1. In other embodiments, the CCL3 receptor is CCR5. In various embodiments, the CCL3 receptor inhibitor useful in the methods of the invention is at least one of a chemical compound, a protein, a peptide, a peptidomemetic, an antibody, a ribozyme, a small molecule chemical compound, or an antisense nucleic acid molecule. In various embodiments, the myeloid neoplasm is at least one of a myeloproliferative disorder, acute myeloid leukemia (AML), chronic myeloid leukemia (CML), chronic myelomonocytic leukemia (CMML) or myelodysplastic syndrome (MDS). In various embodiments, the pathology associated with myeloid neoplasm treatable by the methods of the invention is at least one of osteoclast/osteoblast imbalance, inhibition of osteoblast function, bone loss, disregulation of the hematopoietic stem cell microenvironment, abnormal hematopoietic recovery, pancytopenia, anemia, thrombocytopenia, neutropenia, bicytopenia, or erythrocytopenia. In some embodiments, the subject is a human.

In one embodiment, the invention is a composition comprising a CCL3 inhibitor, where the CCL3 inhibitor is at least one of a chemical compound, a protein, a peptide, a peptidomemetic, an antibody, a ribozyme, a small molecule chemical compound, and an antisense nucleic acid molecule. In another embodiment, the invention is a composition comprising a CCL3 receptor inhibitor, where the CCL3 receptor inhibitor is at least one of a chemical compound, a protein, a peptide, a peptidomemetic, an antibody, a ribozyme, a small molecule chemical compound, and an antisense nucleic acid molecule. In one embodiment, the CCL3 receptor is CCR1. In another embodiment, the CCL3 receptor is CCR5.

In one embodiment, the invention provides a method of treating bone loss in subject. In one embodiment, the method comprises administering a therapeutic effective amount a CCL3 inhibitor or a CCL3 receptor inhibitor to a subject in need thereof. In one embodiment, the method comprises administering a therapeutic effective amount of an agent that inhibits CCL3 signaling.

In one embodiment, the invention provides a method of expanding hematopoietic stem cells (HSCs). In one embodiment, the method comprises contacting a HSC with an effective amount a CCL3 inhibitor or a CCL3 receptor inhibitor. In one embodiment, the method comprises contacting an effective amount of an agent that inhibits CCL3 signaling in the HSC. In one embodiment, the method comprises contacting an effective amount of an agent that inhibits CCL3 signaling in a neighboring cell.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A through 1H, is a series of images depicting murine model of a blast crisis of chronic myeloid leukemia (bcCML). FIG. 1A depicts Murine Stem Cell Virus (MSCV) construct containing BCR/ABL and GFP. FIG. 1B depicts MSCV construct containing Nup98/HoxA9 and YFP. FIG. 1C depicts schematic representation of the transplant strategy used to produce the leukemic mice utilized. FIG. 1D depicts the flow cytometric gating strategy used to identify leukemic cells as GFP and YFP positive. FIG. 1E depicts representative anti-GFP immunohistochemistry of the femur's marrow space at the metaphysis. GFP is visualized by brown staining, with a hematoxylin counterstain. FIG. 1F depicts flow cytometric data representing bcCML cells as a percentage of total marrow mono-nuclear cells. FIG. 1G depicts flow cytometric data representing bcCML cells as a percentage of total spleen, and FIG. 1H depicts total peripheral blood mono-nuclear cells over the course of 10 days. * p≦0.05, ** p≦0.01, *** p≦0.001; n=5 mice per time point. Bar indicates SEM in this and subsequent experiments.

FIG. 2, comprising FIGS. 2A through 2F, is a series of images depicting the results of experiments showing that leukemia decreases osteoblastic number and function. FIGS. 2A and 2B depict osteopontin immunohistochemistry performed on paraffin embedded sections. Low and high power representative images are shown of a naïve femur in FIG. 2A, and a leukemic femur in FIG. 2B. Osteopontin positive cells are stained brown and sections were counterstained with hematoxylin as shown in blue. Arrowheads indicate osteopontin⁺ cells. FIG. 2C depicts quantification of serum osteocalcin measured by ELISA. FIG. 2D depicts real-time RT-PCR quantifying osteocalcin RNA expression in osteoblast-like cells collected from the long bones of normal or leukemic mice at day 6 or 11 and magnetically separated based on CD45 expression (statistical significance was determined compared to naïve mice (day 0); n=5 samples per experimental group). FIGS. 2E and 2F depict CFU-OBs formed per well from (2E) whole marrow after 28 days in culture and (2F) cells collected by collagenase digestion of bone fragments after 15 days in culture. * p≦0.05, ** p≦0.01, *** p≦0.001, * p≦0.05, ** p≦0.01, *** p≦0.001.

FIG. 3, comprising FIGS. 3A through 3I, is a series of images depicting the results of experiments showing that the leukemic environment induces bone loss. FIGS. 3A and 3B depict representative H&E stained Paraffin sections of the distal femur from naïve mice as shown in FIG. 3A and leukemic mice 10 days after transplant as shown in FIG. 3B. FIGS. 3C and 3D depict representative micro-CT images from the metaphysis of the femur from naïve mice as shown in FIG. 3C and leukemic mice as shown in FIG. 3D. FIG. 3E depicts micro-CT analysis of femur trabecular bone volume/total volume and FIG. 3F depicts femur cortical bone volume/total volume. FIG. 3G depicts trabecular number, FIG. 3H depicts trabecular thickness and FIG. 3I depicts trabecular spacing. * p≦0.05, ** p≦0.01; n=4 mice per experiment.

FIG. 4, comprising FIGS. 4A through 4E, is a series of images depicting the results of experiments showing that the leukemic environment mildly and transiently increases osteoclastic numbers in vivo. FIG. 4A depicts serum levels of carboxy-terminal collagen crosslinks (CTX), a marker of bone resorption, measured by ELISA. FIG. 4B depicts representative paraffin sections stained for the osteoclastic marker tartrate resistant acid phosphatase (TRAP). TRAP⁺ cells are pink and highlighted by arrowheads. FIG. 4C depicts quantification of multi-nucleated TRAP positive cells in a 1 mm square area just proximal of the distal growth plate in the femur sections represented by the panels in FIG. 4B. FIG. 4D depicts serum levels of TRACP 5b, the osteoclast specific TRAP enzyme, measured by ELISA. FIG. 4E depicts serum levels of CTX, measured by ELISA. * p≦0.05, ** p≦0.01, *** p≦0.001. Each point indicates an individual mouse in this and subsequent experiments. n=5 mice per experiment. FIG. 4A day 10 n=17.

FIG. 5, comprising FIGS. 5A through 5H, is a series of images depicting the results of experiments showing that leukemia cells do not differentiate into osteoclasts and do not resorb bone. FIG. 5A depicts representative light micrographs of normal cells under osteoclastogenic conditions in vitro. FIG. 5B depicts representative light micrographs of leukemic spleen cells under osteoclastogenic conditions in vitro. Pink cells are positive for TRAP activity. FIG. 5C depicts the quantification of TRAP+ cells in FIGS. 5A and 5B. FIG. 5D depicts low and high power scanning electron micrographs of osteoclasts on bovine bone wafers. FIG. 5E depicts low and high power scanning electron micrographs of leukemia cells on bovine bone wafers. FIG. 5F depicts ELISA quantification of CTX released into culture media during culture of cells with bovine bone wafers. FIGS. 5G and 5H depict representative light micrographs of co-cultures containing osteoblasts and (FIG. 5G) normal marrow cells and (FIG. 5H) leukemic marrow cells. FIG. 5I depicts the quantification of TRAP+ cells formed per well from osteoblastic co-cultures with normal and leukemic marrows. * p≦0.05, *** p≦0.001 n=3-4 mice per treatment group.

FIG. 6, comprising FIGS. 6A through 6G, is a series of images depicting the results of experiments showing that zoledronic acid (ZA) rescues trabecular, but not cortical bone loss. FIG. 6A is a schematic of the treatment schedule of leukemic and normal mice with ZA. Leukemia was initiated on day 0 following 2 weeks of ZA treatment. Injection of ZA is denoted by arrows. FIG. 6B depicts serum CTX levels in mice following 2 weeks of treatment with ZA. FIG. 6C depicts serum CTX levels in normal and leukemic mice following the ZA treatment schedule. FIG. 6D depicts the results of experiments assessing trabecular bone volume/total volume. FIG. 6E depicts the results of experiments evaluating cortical bone volume/total volume. FIG. 6F depicts the results of experiments measuring trabecular number. FIG. 6G depicts the results of experiments assessing trabecular spacing in normal or leukemic mice as quantified by micro-CT analysis following treatment with ZA. ** p≦0.01, *** p≦0.001. n=4 mice per treatment group in FIGS. 6D through 6G.

FIG. 7, comprising FIGS. 7A through 7E, is a series of images depicting the results of experiments showing that CCL-3 expression is increased in malignant cells from leukemic mice. FIG. 7A (top panel) depicts representative wells from CFU-OB cultures stained for alkaline phosphatase activity (pink). FIG. 7A (bottom panel) depicts CCL3 levels in culture media from CFU-OB cultures. FIG. 7B depicts CCL3 protein levels in a murine model of AML compared to normal controls. FIG. 7C depicts relative expression of CCL3 in bone marrow mononuclear cells isolated from whole bone marrow, cells sorted for GFP and YFP expression according to FIG. 1D, and cells liberated from bone fragments by collagenase digestion and magnetically separated based on CD45 cell surface expression. FIG. 7D depicts relative expression of human CCL3 in primitive CD34+CD38−CD123+AML cells compared to normal controls each bar represents a single AML sample normalized to 3 normal controls. FIG. 7E depicts CCL3 protein levels in human AML patient marrow plasma. Each bar represents a single AML marrow sample compared to 7 normal controls. p≦0.05, ** p≦0.01. Each data point represents an individual mouse in FIGS. 7A and 7B and 7D, n=3 mice per group in FIG. 7C.

FIG. 8, comprising FIGS. 8A through 8F, is a series of images depicting the results of experiments assessing the presence of leukemia stem cells (LSC). FIGS. 8A, 8B and 8C depict flow cytometry data quantifying bcCML cells as a percent of total mononuclear cells in the marrow, the spleen, and the peripheral blood, respectively, from mice treated with either vehicle or ZA. FIG. 8D depicts flow cytometry gating strategy to isolate a population enriched for leukemia stem cells (LSC).

FIGS. 8E and 8F depict flow cytometry data quantifying a population of bcCML cells enriched for LSCs as a percent of total mononuclear cells in the marrow and the spleen, respectively. Each figure is a combination of 2 separate experiments. * p≦0.05.

FIG. 9 is a schematic of how AML is initiated in an animal by administering leukemic cells to the animal.

FIG. 10 comprises a series of images demonstrating that CCL3^{-/-} AML cells fail to initiate leukemia in WT recipient mice.

FIG. 11 comprises a series of images demonstrating that CCL3^{-/-} mice display altered trabecular architecture (e.g., Femur BV/TV, Femur Conn. D., Femur Tb.N).

FIG. 12 comprises a series of images demonstrating that CCL3^{-/-} mice display altered trabecular architecture (e.g., Femur Tb.Sp, Femur Tb.Th., Femur Cortical Thickness).

FIG. 13 comprises a series of images demonstrating that CCL3KO mice have a greater number of total marrow phenotypic HSCs.

FIG. 14 comprises a series of images demonstrating that CCL3KO mice have no change in the number of total splenic phenotypic HSCs.

### DETAILED DESCRIPTION

The present invention relates to the discovery that CCL3, through at least one of its receptors CCR1 and CCR5, plays a role in the pathologies associated with myeloid neoplasms. In various embodiments, the pathologies associated with myeloid neoplasms that are treatable by the compositions and methods of the invention described herein include, but are not limited to, at least one of osteoclast/osteoblast imbalance, inhibition of osteoblast function, bone loss, disregulation of the hematopoietic stem cell microenvironment, abnormal hematopoietic recovery, pancytopenia, anemia, thrombocytopenia, neutropenia, bicytopenia, and erythrocytopenia. Interfering with the interaction between CCL3 and its receptors, by targeting at least one of CCL3, CCR1, CCR5, diminishes the effects of pathologies associated with myeloid neoplasms. In various embodiments, the myeloid neoplasm is a myeloproliferative disorder, acute myeloid leukemia (AML), chronic myeloid leukemia (CML), chronic myelomonocytic leukemia (CMML), or myelodysplastic syndrome (MDS).

In one embodiment, the present invention is directed to methods and compositions for treatment, inhibition, prevention, or reduction of a myeloid neoplasm and/or bone loss. In one embodiment, the invention provides compositions and methods for modulating one or more of the level, production, and activity of at least one of CCL3, CCR1, and CCR5.

Accordingly, the invention provides inhibitors (e.g., antagonists) of at least one of CCL3, CCR1, and CCR5. In one embodiment, the inhibitor of at least one of CCL3, CCR1, and CCR5 includes but is not limited to an antibody or a fragment thereof, a peptide, a nucleic acid, small molecule, a chemical compound, and the like.

Another aspect of the present invention comprises a pharmaceutical composition comprising an inhibitor of at least one of CCL3, CCR1, and CCR5. In one embodiment, the composition of the invention can be used in combination with another therapeutic agent.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, or ±10%, or ±5%, or ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type. The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The an antibody in the present invention may exist in a variety of forms where the antigen binding portion of the antibody is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to at least one portion of an intact antibody and refers to the antigenic determining variable regions of an intact antibody. Examples of antibody fragments include, but are not limited to, Fab, Fab′, F(ab′)2, and Fv fragments, linear antibodies, sdAb (either V_{L} or V_{H}), camelid V_{HH} domains, scFv antibodies, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it was derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

The term “inhibit,” as used herein, means to suppress or block an activity or function, for example, about ten percent relative to a control value. Preferably, the activity is suppressed or blocked by 50% compared to a control value, more preferably by 75%, and even more preferably by 95% “Inhibit,” as used herein, also means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “biological sample” as used herein, is intended to include any sample comprising a cell, a tissue, or a bodily fluid in which expression of a nucleic acid or polypeptide can be detected. Examples of such biological samples include but are not limited to blood, lymph, bone marrow, biopsies and smears. Samples that are liquid in nature are referred to herein as “bodily fluids.” Biological samples may be obtained from a patient by a variety of techniques including, for example, by scraping or swabbing an area or by using a needle to obtain bodily fluids. Methods for collecting various body samples are well known in the art.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “heavy chain antibody” or “heavy chain antibodies” comprises immunoglobulin molecules derived from camelid species, either by immunization with a peptide and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of VH (variable heavy chain immunoglobulin) genes from an animal.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding”, can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “DNA” as used herein is defined as deoxyribonucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “progeny” as used herein refers to a descendent or offspring and includes the differentiated or undifferentiated decedent cell derived from a parent cell. In one usage, the term progeny refers to a descendent cell which is genetically identical to the parent. In another use, the term progeny refers to a descendent cell which is genetically and phenotypically identical to the parent. In yet another usage, the term progeny refers to a descendent cell that has differentiated from the parent cell.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

As used herein, “conjugated” refers to covalent attachment of one molecule to a second molecule.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to the discovery that CCL3, through at least one of its receptors CCR1 and CCR5, plays a role in the pathologies associated with myeloid neoplasm. In various embodiments, the pathologies associated with myeloid neoplasm that are treatable by the compositions and methods described herein include, but are not limited to, at least one of osteoclast/osteoblast imbalance, inhibition of osteoblast function, decreased maturation of osteoblastic cells, decreased maturation in osteoblastic cells, bone loss, disregulation of the hematopoietic stem cell microenvironment, abnormal hematopoietic recovery, pancytopenia, anemia, thrombocytopenia, neutropenia, bicytopenia, and erythrocytopenia. Interfering with the interaction between CCL3 and its receptors, by targeting CCL3, CCR1, CCR5, or combinations thereof, diminishes the effects of pathologies associated with myeloid neoplasm. In various embodiments, the myeloid neoplasm is a myeloproliferative disorder, acute myeloid leukemia (AML), chronic myeloid leukemia (CML), chronic myelomonocytic leukemia (CMML), or myelodysplastic syndrome (MDS).

In various embodiments, the present invention includes compositions and methods of treating pathologies associated with myeloid neoplasm by diminishing the expression level, or activity level, of at least one of CCL3, CCR1 and CCR5. In other embodiments, the invention includes compounds and methods for treating pathologies associated with myeloid neoplasm by interfering with the interaction between CCL3 and at least one of its receptors (e.g., CCR1 and CCR5). In still further embodiments, the invention includes compounds and methods for treating pathologies associated with myeloid neoplasm by interfering with signal transduction through at least one of CCR1 and CCR5. Accordingly, the invention includes regulating any target associated with a signal transduction pathway involved with CCL3 interacting with at least one of CCR1 and CCR5.

In one embodiment, the pathology associated with myeloid neoplasm includes marrow microenvironmental changes. In some embodiments, the marrow microenvironmental change includes the disruption of normal hematopoietic stem cell microenvironment support. In other embodiments, the marrow microenvironmental change includes the enhancement of abnormal support of the myeloid neoplasm and neoplastic stem cells. In a further embodiment, the marrow microenvironmental change includes both the disruption of normal hematopoietic stem cell microenvironment support, as well as the enhancement of abnormal support of the myeloid neoplasm and neoplastic stem cells.

### Inhibition of CCL3

In various embodiments, the present invention includes compositions and methods of treating pathologies associated with myeloid neoplasm by diminishing the expression level, or activity level, of CCL3. In other embodiments, the invention includes compounds and methods for treating pathologies associated with myeloid neoplasm by interfering with the interaction between CCL3 and at least one of its receptors (e.g., CCR1 and CCR5).

It would be understood by one skilled in the art, based upon the disclosure provided herein, that a decrease in the level of CCL3 encompasses the decrease of CCL3 expression. Additionally, the skilled artisan would appreciate, once armed with the teachings of the present invention, that a decrease in the level of CCL3 includes a decrease in CCL3 activity. Thus, decreasing the level or activity of CCL3 includes, but is not limited to, decreasing transcription, translation, or both, of a nucleic acid encoding CCL3; and it also includes decreasing any activity of CCL3 as well.

Inhibition of CCL3 can be assessed using a wide variety of methods, including those disclosed herein, as well as methods well-known in the art or to be developed in the future. That is, the routineer would appreciate, based upon the disclosure provided herein, that decreasing the level or activity of CCL3 can be readily assessed using methods that assess the level of a nucleic acid encoding CCL3 (e.g., mRNA) and/or the level of CCL3 protein present in a biological sample.

One skilled in the art, based upon the disclosure provided herein, would understand that the invention is useful in treating pathologies associated with myeloid neoplasm in subjects who have myeloid neoplasm, whether or not the subject also being treated with other medication or chemotherapy. Further, the skilled artisan would further appreciate, based upon the teachings provided herein, that the pathologies associated with myeloid neoplasm treatable by the compositions and methods described herein encompass any pathology associated with myeloid neoplasm where CCL3, CCR1 or CCR5 plays a role.

A CCL3 inhibitor can include, but should not be construed as being limited to, a chemical compound, a protein, a peptide, a peptidomemetic, an antibody, a ribozyme, a small molecule chemical compound, and an antisense nucleic acid molecule (e.g., siRNA, miRNA, etc.). One of skill in the art would readily appreciate, based on the disclosure provided herein, that a CCL3 inhibitor encompasses a chemical compound that decreases the level or activity of CCL3. Additionally, a CCL3 inhibitor encompasses a chemically modified compound, and derivatives, as is well known to one of skill in the chemical arts.

Further, one of skill in the art would, when equipped with this disclosure and the methods exemplified herein, appreciate that a CCL3 inhibitor includes such inhibitors as discovered in the future, as can be identified by well-known criteria in the art of pharmacology, such as the physiological results of inhibition of CCL3 as described in detail herein and/or as known in the art. Therefore, the present invention is not limited in any way to any particular CCL3 inhibitor as exemplified or disclosed herein; rather, the invention encompasses those inhibitors that would be understood by the routineer to be useful as are known in the art and as are discovered in the future.

Further methods of identifying and producing CCL3 inhibitors are well known to those of ordinary skill in the art, including, but not limited, obtaining an inhibitor from a naturally occurring source (i.e., Streptomyces sp., Pseudomonas sp., Stylotella aurantium). Alternatively, a CCL3 inhibitor can be synthesized chemically. Further, the routineer would appreciate, based upon the teachings provided herein, that a CCL3 inhibitor can be obtained from a recombinant organism. Compositions and methods for chemically synthesizing CCL3 inhibitors and for obtaining them from natural sources are well known in the art and are described in the art.

One of skill in the art will appreciate that an inhibitor can be administered as a small molecule chemical, a protein, a nucleic acid construct encoding a protein, an antisense nucleic acid, a nucleic acid construct encoding an antisense nucleic acid, or combinations thereof. Numerous vectors and other compositions and methods are well known for administering a protein or a nucleic acid construct encoding a protein to cells or tissues. Therefore, the invention includes a method of administering a protein or a nucleic acid encoding a protein that is an inhibitor of CCL3. (Sambrook et al., 1989, Molecular Cloning: A Laboratory Manual, Cold Spring Harbor Laboratory, New York; Ausubel et al., 1997, Current Protocols in Molecular Biology, John Wiley & Sons, New York).

One of skill in the art will realize that diminishing the amount or activity of a molecule that itself increases the amount or activity of CCL3 can serve in the compositions and methods of the present invention to decrease the amount or activity of CCL3.

Antisense oligonucleotides are DNA or RNA molecules that are complementary to some portion of an mRNA molecule. When present in a cell, antisense oligonucleotides hybridize to an existing mRNA molecule and inhibit translation into a gene product Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of an antisense oligonucleotide to diminish the amount of CCL3, or to diminish the amount of a molecule that causes an increase in the amount or activity of CCL3, thereby decreasing the amount or activity of CCL3.

Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expressing CCL3, or of a gene expressing a protein that increases the level or activity of CCL3, can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28: 4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

One of skill in the art will appreciate that inhibitors of CCL3 can be administered singly or in any combination. Further, CCL3 inhibitors can be administered singly or in any combination in a temporal sense, in that they may be administered simultaneously, before, and/or after each other. One of ordinary skill in the art will appreciate, based on the disclosure provided herein, that CCL3 inhibitors can be used to treat pathologies associated with myeloid neoplasm, and that an inhibitor can be used alone or in any combination with another inhibitor to effect a therapeutic result.

It will be appreciated by one of skill in the art, when armed with the present disclosure including the methods detailed herein, that the invention is not limited to treatment of a pathology associated with myeloid neoplasm that is already established. Particularly, the pathology need not have manifested to the point of detriment to the subject; indeed, the pathology need not be detected in a subject before treatment is administered. That is, significant pathology associated with myeloid neoplasm does not have to occur before the present invention may provide benefit. Therefore, the present invention includes a method for preventing a pathology associated with myeloid neoplasm in a subject, in that a CCL3 inhibitor, as discussed previously elsewhere herein, can be administered to a subject prior to the onset of a pathology, thereby preventing the pathology. The preventive methods described herein also include the treatment of a subject that is in remission for the prevention of a recurrence.

One of skill in the art, when armed with the disclosure herein, would appreciate that the prevention of pathology associated myeloid neoplasm encompasses administering to a subject a CCL3 inhibitor as a preventative measure against a pathology associated with myeloid neoplasm. As more fully discussed elsewhere herein, methods of decreasing the level or activity of CCL3 encompass a wide plethora of techniques for decreasing not only CCL3 activity, but also for decreasing expression of a nucleic acid encoding CCL3.

Further, the compositions and methods described herein for treating or preventing pathologies associated with myeloid neoplasm in vivo in a subject in need thereof, can also be used in an ex vivo context. By way of nonlimiting examples, the compositions and methods described herein can be used to treat autologous stem cell products ex vivo, before their transplantation into the subject, or to treat bone marrow mesenchymal stem cells ex vivo, before their transplantation into the subject.

Additionally, as disclosed elsewhere herein, one skilled in the art would understand, once armed with the teaching provided herein, that the present invention encompasses a method of preventing a wide variety of diseases, disorders and pathologies where a decrease in expression and/or activity of CCL3 mediates, treats or prevents the disease, disorder or pathology. Methods for assessing whether a disease relates to increased levels or activity of CCL3 are known in the art. Further, the invention encompasses treatment or prevention of such diseases discovered in the future.

The invention encompasses administration of an inhibitor of CCL3 to practice the methods of the invention; the skilled artisan would understand, based on the disclosure provided herein, how to formulate and administer the appropriate CCL3 inhibitor to a subject. Indeed, the successful administration of the CCL3 inhibitor has been reduced to practice as exemplified herein. However, the present invention is not limited to any particular method of administration or treatment regimen.

### Inhibition of a Receptor of CCL3

In various embodiments, the present invention includes compositions and methods of treating pathologies associated with myeloid neoplasm by diminishing the expression level, or activity level, of at least one of the receptors of CCL3 (e.g., CCR1 and CCR5). In other embodiments, the invention includes compounds and methods for treating pathologies associated with myeloid neoplasm by interfering with the interaction between at least one of CCR1 and CCR5, and their ligand, CCL3. In still further embodiments, the invention includes compounds and methods for treating pathologies associated with myeloid neoplasm by interfering with signal transduction through at least one of CCR1 and CCR5.

It would be understood by one skilled in the art, based upon the disclosure provided herein, that a decrease in the level of at least one CCL3 receptor encompasses the decrease in expression at least one CCL3 receptor. Additionally, the skilled artisan would appreciate, once armed with the teachings of the present invention, that a decrease in the level of at least one CCL3 receptor includes a decrease in the activity of at least one CCL3 receptor. Thus, decreasing the level or activity of at least one CCL3 receptor includes, but is not limited to, decreasing transcription, translation, or both, of a nucleic acid encoding a CCL3 receptor; and it also includes decreasing any activity of a CCL3 receptor as well.

Inhibition of a CCL3 receptor can be assessed using a wide variety of methods, including those disclosed herein, as well as methods well-known in the art or to be developed in the future. That is, the routineer would appreciate, based upon the disclosure provided herein, that decreasing the level or activity of a CCL3 receptor can be readily assessed using methods that assess the level of a nucleic acid encoding a CCL3 receptor (e.g., mRNA) and/or the level of a CCL3 receptor protein present in a biological sample.

One skilled in the art, based upon the disclosure provided herein, would understand that the invention is useful in treating pathologies associated with myeloid neoplasm in subjects who have myeloid neoplasm, whether or not the subject also being treated with other medication or chemotherapy. Further, the skilled artisan would further appreciate, based upon the teachings provided herein, that the pathologies associated with myeloid neoplasm treatable by the compositions and methods described herein encompass any pathology associated with myeloid neoplasm where CCL3, CCR1 or CCR5 plays a role.

A CCL3 receptor inhibitor can include, but should not be construed as being limited to, a chemical compound, a protein, a peptide, a peptidomemetic, an antibody, a ribozyme, a small molecule chemical compound, and an antisense nucleic acid molecule (e.g., siRNA, miRNA, etc.). One of skill in the art would readily appreciate, based on the disclosure provided herein, that a CCL3 receptor inhibitor encompasses a chemical compound that decreases the level or activity of a CCL3 receptor. Additionally, a CCL3 receptor inhibitor encompasses a chemically modified compound, and derivatives, as is well known to one of skill in the chemical arts.

By way of a non-limiting example, a CCL3 receptor inhibitor of the invention includes a CCR1 inhibitor. CCR1 inhibitors include CCR1 antagonists developed for the treatment of chronic inflammatory including but is not limited to MLN 3897 (Millennium), MLN3701 (Millennium), CCX354-C (ChemoCentryx), BX471 (Berlex), CP-481,715 (Pfizer), and the like. However, the invention should not be limited to only these CCR1 antagonists listed herein. Rather, any CCR1 antagonist is included in the invention.

By way of a non-limiting example, a CCL3 receptor inhibitor of the invention includes a CCR5 inhibitor. CCR5 inhibitors include CCR5 antagonists developed for the treatment of HIV including but is not limited to Maraviroc (Pfizer), Vicriviroc (Schering-Plough), Cenicriviroc (Takeda), PRO 140 (Cytodyn), and the like. However, the invention should not be limited to only these CCR5 antagonists listed herein. Rather, any CCR5 antagonist is included in the invention.

Further, one of skill in the art would, when equipped with this disclosure and the methods exemplified herein, appreciate that a CCL3 receptor inhibitor includes such inhibitors as discovered in the future, as can be identified by well-known criteria in the art of pharmacology, such as the physiological results of inhibition of a CCL3 receptor as described in detail herein and/or as known in the art. Therefore, the present invention is not limited in any way to any particular CCL3 receptor inhibitor as exemplified or disclosed herein; rather, the invention encompasses those inhibitors that would be understood by the routineer to be useful as are known in the art and as are discovered in the future.

Further methods of identifying and producing CCL3 receptor inhibitors are well known to those of ordinary skill in the art, including, but not limited, obtaining an inhibitor from a naturally occurring source (i.e., Streptomyces sp., Pseudomonas sp., Stylotella aurantium). Alternatively, a CCL3 receptor inhibitor can be synthesized chemically. Further, the routineer would appreciate, based upon the teachings provided herein, that a CCL3 receptor inhibitor can be obtained from a recombinant organism. Compositions and methods for chemically synthesizing a CCL3 receptor inhibitor and for obtaining them from natural sources are well known in the art and are described in the art.

One of skill in the art will appreciate that an inhibitor can be administered as a small molecule chemical, a protein, a nucleic acid construct encoding a protein, an antisense nucleic acid, a nucleic acid construct encoding an antisense nucleic acid, or combinations thereof. Numerous vectors and other compositions and methods are well known for administering a protein or a nucleic acid construct encoding a protein to cells or tissues. Therefore, the invention includes a method of administering a protein or a nucleic acid encoding a protein that is an inhibitor of a CCL3 receptor. (Sambrook et al., 1989, Molecular Cloning: A Laboratory Manual, Cold Spring Harbor Laboratory, New York; Ausubel et al., 1997, Current Protocols in Molecular Biology, John Wiley & Sons, New York).

One of skill in the art will realize that diminishing the amount or activity of a molecule that itself increases the amount or activity of a CCL3 receptor can serve in the compositions and methods of the present invention to decrease the amount or activity of a CCL3 receptor.

Antisense oligonucleotides are DNA or RNA molecules that are complementary to some portion of an mRNA molecule. When present in a cell, antisense oligonucleotides hybridize to an existing mRNA molecule and inhibit translation into a gene product Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of an antisense oligonucleotide to diminish the amount of a CCL3 receptor, or to diminish the amount of a molecule that causes an increase in the amount or activity of a CCL3 receptor, thereby decreasing the amount or activity of a CCL3 receptor.

Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expressing a CCL3 receptor, or of a gene expressing a protein that increases the level or activity of a CCL3 receptor, can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28: 4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

One of skill in the art will appreciate that inhibitors of a CCL3 receptor can be administered singly or in any combination. Further, CCL3 receptor inhibitors can be administered singly or in any combination in a temporal sense, in that they may be administered simultaneously, before, and/or after each other. One of ordinary skill in the art will appreciate, based on the disclosure provided herein, that a CCL3 receptor inhibitor can be used to treat pathologies associated with myeloid neoplasm, and that an inhibitor can be used alone or in any combination with another inhibitor to effect a therapeutic result.

It will be appreciated by one of skill in the art, when armed with the present disclosure including the methods detailed herein, that the invention is not limited to treatment of a pathology associated with myeloid neoplasm that is already established. Particularly, the pathology need not have manifested to the point of detriment to the subject; indeed, the pathology need not be detected in a subject before treatment is administered. That is, significant pathology associated with myeloid neoplasm does not have to occur before the present invention may provide benefit. Therefore, the present invention includes a method for preventing a pathology associated with myeloid neoplasm in a subject, in that a CCL3 receptor inhibitor, as discussed previously elsewhere herein, can be administered to a subject prior to the onset of a pathology, thereby preventing the pathology.

One of skill in the art, when armed with the disclosure herein, would appreciate that the prevention of pathology associated myeloid neoplasm encompasses administering to a subject a CCL3 receptor inhibitor as a preventative measure against a pathology associated with myeloid neoplasm. As more fully discussed elsewhere herein, methods of decreasing the level or activity of a CCL3 receptor encompass a wide plethora of techniques for decreasing not only a CCL3 receptor activity, but also for decreasing expression of a nucleic acid encoding a CCL3 receptor.

Additionally, as disclosed elsewhere herein, one skilled in the art would understand, once armed with the teaching provided herein, that the present invention encompasses a method of preventing a wide variety of diseases, disorders and pathologies where a decrease in expression and/or activity of a CCL3 receptor mediates, treats or prevents the disease, disorder or pathology. Methods for assessing whether a disease relates to increased levels or activity of a CCL3 receptor are known in the art. Further, the invention encompasses treatment or prevention of such diseases discovered in the future.

The invention encompasses administration of an inhibitor of a CCL3 receptor to practice the methods of the invention; the skilled artisan would understand, based on the disclosure provided herein, how to formulate and administer the appropriate CCL3 receptor inhibitor to a subject. Indeed, the successful administration of the CCL3 receptor inhibitor has been reduced to practice as exemplified herein. However, the present invention is not limited to any particular method of administration or treatment regimen.

### Therapeutic Application

The present invention includes an inhibitor of any one or more of CCL3 or CCL3 receptor or functional equivalent of any of these proteins. The present invention includes a method of treating or preventing cancer in a mammal comprising administering an inhibitor of the invention to the mammal in need thereof. The present invention also includes a method of treating or preventing bond loss in a mammal comprising administering an inhibitor of the invention to the mammal in need thereof. The present invention includes a method of expanding HSC using an inhibitor of the invention.

In one embodiment, the mammal has a type of cancer which expresses a tumor-specific antigen. In accordance with the present invention, an antigenic composition can be made which comprises a tumor-specific antigen sequence component. In such cases, the inhibitor of the invention is administered in combination with an immunostimulatory protein to a patient in need thereof, resulting in an improved therapeutic outcome for the patient, evidenced by, e.g., a slowing or diminution of the growth of cancer cells or a solid tumor which expresses the tumor-specific antigen, or a reduction in the total number of cancer cells or total tumor burden.

The disorder or disease can be treated by administration of an inhibitor of one or more of CCL3 or CCL3 receptor, or their functional equivalents optionally in combination with an antigen (vaccine) to a patient in need thereof.

In another embodiment, the compounds of the present invention may be used in combination with existing therapeutic agents used to treat cancer. In some instances, the compounds of the invention may be used in combination these therapeutic agents to enhance the antitumor effect of the therapeutic agent.

In order to evaluate potential therapeutic efficacy of the compounds of the invention in combination with the antitumor therapeutics described elsewhere herein, these combinations may be tested for antitumor activity according to methods known in the art.

In one aspect, the present invention contemplates that the inhibitors of the invention may be used in combination with a therapeutic agent such as an anti-tumor agent including but not limited to a chemotherapeutic agent, an anti-cell proliferation agent or any combination thereof.

The invention should not limited to any particular chemotherapeutic agent. Rather, any chemotherapeutic agent can be linked to the antibodies of the invention. For example, any conventional chemotherapeutic agents of the following non-limiting exemplary classes are included in the invention: alkylating agents; nitrosoureas; antimetabolites; antitumor antibiotics; plant alkyloids; taxanes; hormonal agents; and miscellaneous agents.

Alkylating agents are so named because of their ability to add alkyl groups to many electronegative groups under conditions present in cells, thereby interfering with DNA replication to prevent cancer cells from reproducing. Most alkylating agents are cell cycle non-specific. In specific aspects, they stop tumor growth by cross-linking guanine bases in DNA double-helix strands. Non-limiting examples include busulfan, carboplatin, chlorambucil, cisplatin, cyclophosphamide, dacarbazine, ifosfamide, mechlorethamine hydrochloride, melphalan, procarbazine, thiotepa, and uracil mustard.

Anti-metabolites prevent incorporation of bases into DNA during the synthesis (S) phase of the cell cycle, prohibiting normal development and division. Non-limiting examples of antimetabolites include drugs such as 5-fluorouracil, 6-mercaptopurine, capecitabine, cytosine arabinoside, floxuridine, fludarabine, gemcitabine, methotrexate, and thioguanine.

There are a variety of antitumor antibiotics that generally prevent cell division by interfering with enzymes needed for cell division or by altering the membranes that surround cells. Included in this class are the anthracyclines, such as doxorubicin, which act to prevent cell division by disrupting the structure of the DNA and terminate its function. These agents are cell cycle non-specific. Non-limiting examples of antitumor antibiotics include dactinomycin, daunorubicin, doxorubicin, idarubicin, mitomycin-C, and mitoxantrone.

Plant alkaloids inhibit or stop mitosis or inhibit enzymes that prevent cells from making proteins needed for cell growth. Frequently used plant alkaloids include vinblastine, vincristine, vindesine, and vinorelbine. However, the invention should not be construed as being limited solely to these plant alkaloids.

The taxanes affect cell structures called microtubules that are important in cellular functions. In normal cell growth, microtubules are formed when a cell starts dividing, but once the cell stops dividing, the microtubules are disassembled or destroyed. Taxanes prohibit the microtubules from breaking down such that the cancer cells become so clogged with microtubules that they cannot grow and divide. Non-limiting exemplary taxanes include paclitaxel and docetaxel.

Hormonal agents and hormone-like drugs are utilized for certain types of cancer, including, for example, leukemia, lymphoma, and multiple myeloma. They are often employed with other types of chemotherapy drugs to enhance their effectiveness. Sex hormones are used to alter the action or production of female or male hormones and are used to slow the growth of breast, prostate, and endometrial cancers Inhibiting the production (aromatase inhibitors) or action (tamoxifen) of these hormones can often be used as an adjunct to therapy. Some other tumors are also hormone dependent. Tamoxifen is a non-limiting example of a hormonal agent that interferes with the activity of estrogen, which promotes the growth of breast cancer cells.

Miscellaneous agents include chemotherapeutics such as bleomycin, hydroxyurea, L-asparaginase, and procarbazine that are also useful in the invention.

An anti-cell proliferation agent can further be defined as an apoptosis-inducing agent or a cytotoxic agent. The apoptosis-inducing agent may be a granzyme, a Bcl-2 family member, cytochrome C, a caspase, or a combination thereof. Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or a combination thereof. In other specific aspects, the Bcl-2 family member is, for example, Bax, Bak, Bcl-Xs, Bad, Bid, Bik, Hrk, Bok, or a combination thereof.

In additional aspects, the caspase is caspase-1, caspase-2, caspase-3, caspase-4, caspase-5, caspase-6, caspase-7, caspase-8, caspase-9, caspase-10, caspase-11, caspase-12, caspase-13, caspase-14, or a combination thereof. In specific aspects, the cytotoxic agent is TNF-α, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriaminepentaacetic acid, irofulven, Diptheria Toxin, mitogillin, ricin, botulinum toxin, cholera toxin, saporin 6, or a combination thereof.

In some embodiments, an effective amount of a compound of the invention and a therapeutic agent is a synergistic amount. As used herein, a “synergistic combination” or a “synergistic amount” of a compound of the invention and a therapeutic agent is a combination or amount that is more effective in the therapeutic or prophylactic treatment of a disease than the incremental improvement in treatment outcome that could be predicted or expected from a merely additive combination of (i) the therapeutic or prophylactic benefit of the compound of the invention when administered at that same dosage as a monotherapy and (ii) the therapeutic or prophylactic benefit of the therapeutic agent when administered at the same dosage as a monotherapy.

### Pharmaceutical Compositions

Compounds which are identified using any method described herein as potential useful compounds for treatment and/or prevention of myeloid neoplasm can be formulated and administered to a subject for treatment of myeloid neoplasm disclosed herein are now described.

The invention encompasses the preparation and use of pharmaceutical compositions comprising a compound useful for treatment of myeloid neoplasm disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically-acceptable carrier” means a chemical composition with which an appropriate inhibitor thereof, may be combined and which, following the combination, can be used to administer the appropriate inhibitor thereof, to a subject.

The pharmaceutical compositions useful for practicing the invention may be administered to deliver a dose of between about 0.1 ng/kg/day and 100 mg/kg/day.

In various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate inhibitor, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer an appropriate inhibitor thereof, according to the methods of the invention.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods of the invention may be prepared, packaged, or sold in formulations suitable for oral, rectal, vaginal, parenteral, topical, pulmonary, intranasal, buccal, intravenous, ophthalmic, intrathecal and other known routes of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from about 0.01 mg to 20 about 100 g per kilogram of body weight of the animal. While the precise dosage administered will vary depending upon any number of factors, including, but not limited to, the type of animal and type of disease state being treated, the age of the animal and the route of administration. Preferably, the dosage of the compound will vary from about 1 mg to about 100 mg per kilogram of body weight of the animal. More preferably, the dosage will vary from about 1 μg to about 1 g per kilogram of body weight of the animal. The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Functional Inhibition of Osteoblastic Cells in an In Vivo Mouse Model of Myeloid Leukemia

Pancytopenia is the cause of significant morbidity in leukemia, and yet the mechanisms by which leukemia causes anemias remain poorly understood. It is shown that induction of myeloid leukemia in an in vivo immunocompetent non-irradiated murine microenvironment induces severe functional inhibition of osteoblastic cells even when the burden of disease is relatively low and when leukemic cells are undetectable in blood. Therefore, osteoblastic damage by leukemia may be due to specific leukemic-initiated interactions rather than as a consequence of systemic disease.

Osteoblastic inhibition in leukemia resulted in decreased bone formation and net bone loss, particularly in cortical bone, where inhibition of osteoclast activity could not compensate for the leukemic-dependent bone loss. While there is little data reporting the effects on bone in adult AML, these data are consistent with findings at diagnosis in pediatric acute leukemia, where decreased markers of bone formation prior to corticosteroid treatment have been documented in numerous studies (2007, Sala and Barr, Cancer 109:1420-1431; 2008, Sinigaglia et al., J Pediatr Orthop. 28:20-28). This disruption in bone formation may be reversible as, in spite of corticosteroid treatment in this disease, bone formation markers improve with reduction of disease burden after chemotherapy (1998, Crofton et al., J Clin Endocrinol Metab. 83:3121-3129).

Bone turnover is a closely regulated event in which bone formation and bone resorption are tightly coupled. In the setting of dramatic loss of bone formation, a compensatory loss of osteoclastic bone resorption would be expected. In contrast, there was a mild and transient increase in osteoclastic cells in mice with leukemia. Moreover, leukemic-induced trabecular bone loss was completely blocked by osteoclast inhibition. Therefore, leukemia initiation results in uncoupling of bone formation and bone resorption. This effect is likely responsible for the trabecular loss, and may be important in the early stages of disease. The decrease in osteoclastic number seen at later time points may be due, in part, to the leukemia-induced block in hematopoietic differentiation that could limit the pool of hematopoietic osteoclast precursors. This result also indicates a potential role for osteoclastic inhibition, particularly at time of recurrence where the percent of blasts present in the marrow is relatively low and hematopoietic differentiation is preserved.

The mechanisms by which AML inhibits osteoblastic cells may include both direct and indirect actions of leukemia on their microenvironment. Receptors for CCL3 (CCR1 and CCR5) are present on osteoblastic cells (2003, Abbas et al., Cytokine 22:33-41; 2005, Yano et al., Endocrinology 146:2324-2335) and recent data have demonstrated the inhibitory effects of CCL3 on both murine and human osteoblastic cells (2011, Vallet et al., Leukemia). The findings disclosed herein reveal that CCL3 is highly expressed both by the tested leukemic model as well as in human AML. This chemokine also has strong pro-osteoclastic effects (2011, Han et al., Blood 97:3349-3353; 2003, Lentzsch et al., Blood 101:3568-357) that may at least in part explain the transient increase in TRAP+ cells described herein, consistent with the uncoupling of bone formation and bone resorption described herein. The data described herein suggest a therapeutic approach in which CCL3 inhibition may be used to ameliorate osteoblastic dysfunction and accelerate recovery of normal hematopoiesis in the setting of leukemia treatment. The findings described here are consistent with the concept that leukemia disrupts the normal marrow microenvironment, and particularly targets cells that have been demonstrated to support and regulate HSCs.

The materials and methods employed in these experiments are now described.

### Materials and Methods

Mice

The Institutional Animal Care and Use Committee at the University of Rochester School of Medicine and Dentistry approved all animal studies.

Patient Samples

Peripheral blood and marrow aspirates were collected from patients with AML and from healthy volunteer donors. Marrow aspirates were obtained from the posterior iliac crest. Blood and marrow plasma was isolated by centrifugation and analyzed for protein levels by ELISA. Bone marrow and peripheral blood mononuclear cells were isolated and CD34+CD38− normal and leukemic cells were isolated as previously described (Majeti et al., 2009, Proc Natl Acad Sci USA 106:3396-3401). Relative gene expression was performed using real time quantitative PCR. All patients and volunteers provided written, informed consent in accordance with the Declaration of Helsinki on protocols approved by the Research Subjects Review Board of the University of Rochester.

Model of AML

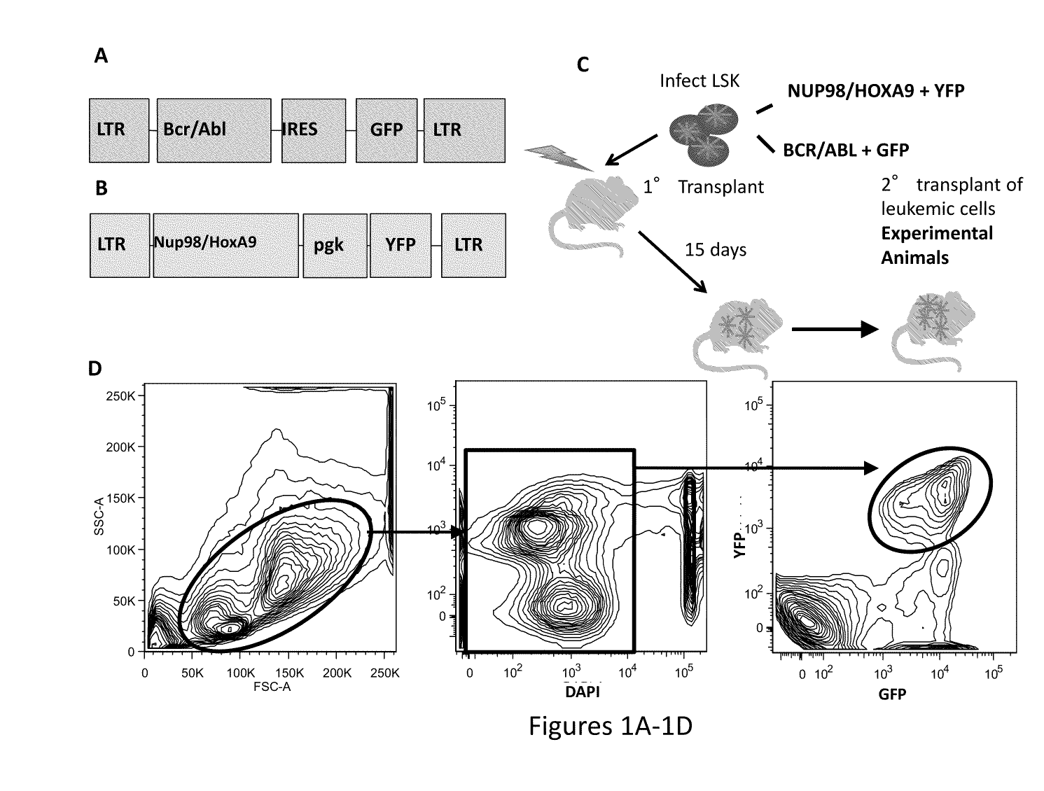

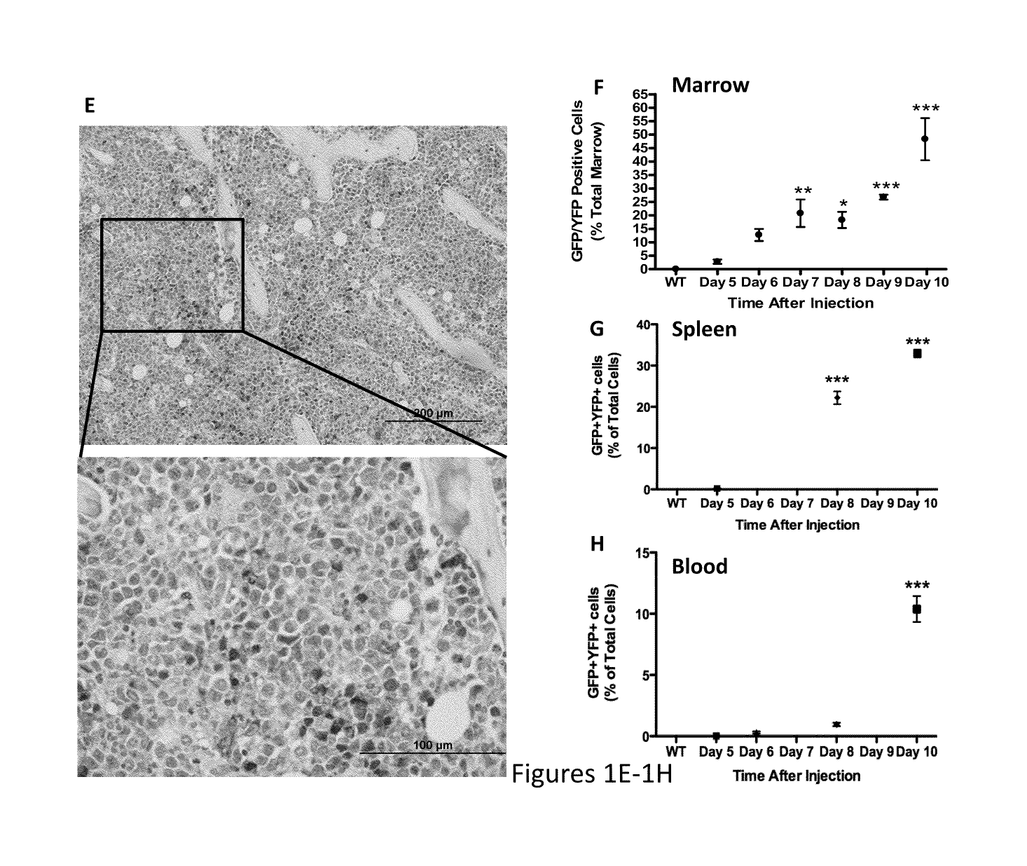

The MSCV-BCR/ABL-IRES-GFP, and MSCV-Nup98/HoxA9-YFP vectors as depicted in FIGS. 1A and 1B were previously described (Neering et al., 2007, Blood 110:2578-2585). Marrow cells from 6-8 week old male CD45.1 mice were enriched for hematopoietic stem and progenitor cells (HSPCs), by FACS sorting to purify the population of lineage negative, sca-1⁺, c-kit⁺ (LSK) cells. LSK cells were infected with both viral vectors simultaneously as previously described (Neering et al., 2007, Blood 110:2578-2585). Six-eight week old male C57bl/6 primary recipients were sub-lethally irradiated (6Gy) using a 137Cs source of radiation (GAMMACELL-40). Immediately following irradiation the mice were injected by tail vein with 2×10⁴ leukemic cells in 0.1 mLs of phosphate buffered saline (PBS) containing 2% heat inactivated fetal bovine serum (FACS buffer). After 15 days the spleens of primary recipient mice were harvested and crushed using the plunger of a 3 mL syringe. The resulting cell suspension was strained using a 40-μM pore size cell strainer, resuspended in Cryostor CS10 (Biolife Solutions) at a concentration of 2×10⁷ cells/mL and cryogenically stored in liquid nitrogen. To induce leukemia in animals used for all described experiments cells were thawed and 2×10⁵ cells in 0.1 mLs FACS buffer were injected by tail vein into non-irradiated 6-8 week old male C57bl/6 mice. These are referred to as leukemic mice, and were sacrificed at the times after secondary transplantation as described for each experiment. Normal controls were always age and sex-matched to the leukemic mice in the same experiment.

Marrow, Spleen, and Peripheral Blood Cell Collection

For hematopoietic analysis, marrow cells were flushed from the long bones of the hindlimbs of mice using a 25 gauge needle. Spleen cells were collected by crushing the spleen inside of a 40 μm cell strainer. Peripheral blood cells were collected by sub-mandibular bleeds followed by incubation for 20 minutes at room temperature in 2% 500,000 molecular weight dextran to precipitate the red blood cells.

Flow Cytometric Analysis and Fluorescent Activated Sorting

Using cells collected as previously described red blood cells (RBCs) were lysed in 1 ml RBC lysis buffer (156 mM NH4Cl, 127 μM EDTA and 12 mM NaHCO₃) for 5 minutes at room temperature, 1×10⁷ cells were suspended in 100 μL FACS buffer and stained with appropriate antibodies. The cells were washed and data was collected on a LSR-II (Beckton Dickson). The data was analyzed using FlowJo software (Tree Star). For sorting, cells were prepared as described for flow cytometric analysis and GFP+/YFP+ cells were sorted using a FACS Aria cell sorter (Beckton Dickson) into FACS buffer.

Histology and Immunohistochemistry

Hind limbs were collected, cleaned of soft tissue, fixed in 10% neutral buffered formalin for 48 hours, and decalcified in 14% EDTA for 10 days. Tissues were then processed and embedded in paraffin, 5 μm thick sections were cut and used for hematoxylin and eosin staining (H&Es), immunohistochemistry or TRAP staining Immunohistochemical staining for GFP and OPN utilized the monoclonal JL-8 antibody (Clontech, 632380) and the AKm2A1 antibody (Santa Cruz biotechnology, Inc. sc-21742) respectively. Both immunohistochemical stains were performed using the M.O.M. kit (Vector Laboratories, Inc. PK-2200) and were counterstained with hematoxylin. TRAP staining was performed as previously reported 20 and counterstained with fast-green.

Osteoblastic Cell Collection from Long Bones

Osteoblastic cells were collected from the long bones of the hind limbs according to a previously described protocol (2010, Chitteti et al., Blood 115:3239-3248). In brief, the long bones of the hindlimbs were cleaned of soft tissue, and the bone marrow was flushed with a 25-gauge needle and discarded. The resulting cleaned and flushed bones were cut into <1 mm fragments and digested twice in collagenase (Stem Cell Technologies, 7902) for 30 and 60 minutes sequentially. Remaining fragments were removed from the collected digest by passing through a 40 μm pore size cell strainer. The cells obtained were seeded in 6-well cell culture dishes at 1×10⁶ cells per well for cell culture and bone nodule formation, or magnetically separated according to CD45 expression using the IMagnet system (BD biosciences, 552311) and a biotinylated CD45 antibody (ebioscience, 13-0451-82).

Bone Nodule Assay

Following 4 days in culture, media was changed to mineralizing media containing 50 μg/ml L-Ascorbic acid 2-phosphate (Sigma), and 10 mM Glycerol 2-phosphate disodium salt hydrate (Sigma) were added. At specified times following the addition of mineralizing media (see figure legends), cells were fixed with 10% neutral buffered formalin for 30 minutes followed by detection of alkaline phosphatase activity using a staining buffer containing 100 mM Tris-HCL (Sigma), 0.005% w/v Naphthol AS MX-PO4 (Sigma), and 0.03% w/v Red Violet LB salt (Sigma), with a final pH of 8.3 for 45 minutes. Following alkaline phosphatase staining, cells were von Kossa stained (2.5% w/v AgNO3 (Sigma) for 30 minutes).

Osteoprogenitor Cultures from Marrow

Whole marrow flushed from the long bones of normal or leukemic mice was sorted as described for GFP-/YFP-cells and seeded in 6-well tissue culture dishes at 4×10⁶ cells per well in α-MEM containing 10% FBS, 100 U/ml penicillin and 100 μg/ml streptomycin (Gibco) and then used for bone nodule assays.

MicroCT Analysis

The right hindlimb of each mouse was fixed in 10% Formalin for 48 hours, and then stored in 70% ethanol at 4° C. MicroCT analysis was performed as previously described (2009, Frisch et al., Blood 114:4054-4063).

ELISAs

All ELISAs were performed as indicated by the manufacturer's instructions. CTX measurements were performed using the RatLaps ELISA (AC-06F1, Immunodiagnostic Systems Inc.). TRACP 5b measurement was performed using Mouse TRAP ELISA (SB-TR103, Immunodiagnostic Systems Inc.). Osteocalcin measurement performed using the Mouse Osteocalcin EIA kit (BT-470, Biomedical Technologies). Bovine CTX from bone wafer culture media was measured using the Crosslaps® for Culture ELISA (AC-07F1, Immunodiagnostic Systems Inc.). Murine CCL3 protein measurement was performed using the Mouse CCL3/MIP-1 alpha Quantikine ELISA Kit (MMA00, R&D systems). Human CCL3 protein measurement was performed using the Human CCL3/MIP-1 alpha Quantikine ELISA Kit (DMA00, R&D systems).

Zoledronic Acid Treatment

Mice were given a 0.25 mg/kg intraperitoneal injection of zoledronic acid (ZA) bi-weekly for 2 weeks prior to induction of disease and throughout the course of the disease.

Osteoclastogenic Cultures

Spleen cells were collected and red blood cells were lysed as previously described. The remaining cells were cultured at a concentration of 8.75×10 cells/ml in alpha Minimum Essential Medium (α-MEM) containing 10% FBS, 100 U/ml penicillin, 100 μg/ml streptomycin (Gibco) (complete α-MEM), and 30 ng/ml M-CSF (R&D systems) for 2 days. Ten ng/ml RANKL (R&D systems) was added to the media and the cultures were continued for an additional 4 days and then TRAP stained. For osteoblastic co-cultures, osteoblastic cells were isolated as described. An osteoblast feeder layer was established by culturing 1.4×10⁴ cells/well in 96 well plates in complete α-MEM supplemented with 1×10⁻⁹ M 1.25 dihydroxyvitamin D3 (Sigma, D1530). After 2 days 8.75×10⁴ whole marrow cells from either normal or leukemic mice were added to each well. Half media changes were performed every 2 days. Cultures were ended and TRAP stained after 7 days.

For cultures utilizing bovine bone wafers, an IsoMet low speed saw with a diamond blade was used to cut 300 μm thick wafers of bone from a 4 mm square pillar of devitalized bovine cortical bone to produce 300 μm×4 mm×4 mm bone wafers.

Analysis of Relative Gene Expression by Real-Time RT-PCR

Total mRNA was extracted using the RNeasy kit (Qiagen) according to manufacturer's instructions. The Quantitect Reverse Transcription kit (Qiagen) was used to transcribe cDNA which was then diluted 1:50 in water, combined with SYBR Green PCR master mix (BioRad) and amplified using MyiQ Single Color PCR detection systems and software under the following conditions: 95° C. for 3 minutes followed by 40 cycles of 95° C. for 15 seconds and 58° C. (Osteocalcin) or 60° C. (CCL-3) for 30 seconds. Data were analyzed using the relative standard curve method, normalized to β-actin.

| | |
|---|---|
| Murine β-actin | SEQ ID NO: 1 |
| | 5′ primer: GCCACTGCCGCATCCTCTT; |
| | SEQ ID NO: 2 |
| | 3′ primer: GGAACCGCTCGTTGCCAATAG; |
| Murine | SEQ ID NO: 3 |
| Osteocalcin | 5′ primer: CCGCCTACAAACGCATCTACG; |
| | SEQ ID NO: 4 |
| | 3′ primer :GAGAGAGGACAGGGAGGATCAAG; |
| Murine CCL3 | SEQ ID NO: 5 |
| | 5′ primer :AAGGATACAAGCAGCAGCGAGTA; |
| | SEQ ID NO: 6 |
| | 3′ primer :TGCAGAGTGTCATGGTACAGAGAA; |

Human gene expression was determined using Taqman technology (Applied Biosystems, Foster City, Calif.). Specific Taqman probes were obtained from Applied Biosystems: CCL3 (Hs00234142_m1) and GAPDH (Hs02758991_g1) cDNA amplification was performed according to manufacturer's specifications using a LightCycler 480 II (Roche).

Co-Cultures and Conditioned Media

CFU-OB cultures were started as described elsewhere herein. After 4 days in culture osteogenic media was added and 1×10⁶ marrow cells from either normal or leukemic animals were added to the cultures. In addition separate cultures containing normal CFU-OB cultures and 1×10⁶ bone marrow cells from leukemic mice were used to produce conditioned media that was added to normal CFU-OB cultures starting on the same day as the bone marrow cells from leukemic mice were added to the co-cultures.

Scanning Electron Microscopy

Bovine bone wafers were removed from culture media and fixed with 2.5% glutaraldehyde in 0.1M sodium cacodylate buffer for 24 hours at 4° C. The next day the wafers were postfixed in cacodylate buffered 1.0% osmium tetroxide, processed through a graded series of ethanol to 100% (x3), processed into a series of mixtures of 100% ethanol and hexamethyldisilazane (HMDS) before transitioning to 100% HMDS (x3). The last exchange of 100% HMDS covering the wafers was allowed to evaporate at room temperature overnight in an uncovered 12 well plate in a fume hood. The dried wafers were mounted onto aluminum stubs, sputter coated for 90 seconds with gold, and imaged using a Zeiss Supra 40VP Field Emission scanning electron microscope.

Statistical Analysis

For quantitative assays, treatment groups were reported as mean plus or minus SEM and compared using the Student t test in GraphPad Prism, Version 5.0b (GraphPad Software). Statistical significance was established at P less than or equal to 0.05.

The results of the experiments are now described.

An Immunocompetent In Vivo Model where Progression of Leukemia is Identified First in Marrow, then Spleen and Peripheral Blood

A previously characterized model of acute leukemia was used in the experiments described herein (FIG. 1A-C) (2010, Neering et al., Blood 110:2578-2585; 2002, Dash et al., Proc Natl Acad Sci USA. 99:7622-7627). Leukemic cells can be identified and quantified based on their expression of Green Fluorescent Protein (GFP) and Yellow Fluorescent Protein (YFP) (FIG. 1D). In this model, secondary transplantation of the leukemia does not require irradiation of the recipient mice. Since the goal was to study the intact microenvironment, the secondary transplantation recipients (FIG. 1D) were chosen as experimental mice (heretofore designated as leukemic mice), and as controls, sex and age-matched normal mice. Leukemic mice have rapid progression of disease that results in the accumulation of leukemic cells in the marrow identified by morphology, immunohistochemistry (FIG. 1E) and flow cytometric analysis (FIG. 1F), though total cellularity of the marrow is unchanged (29.94±2.48 vs 27.18±2.69, mono-nuclear marrow cells/hindlimb, Normal vs Leukemic, n=13 from 3 separate experiments p=0.4595). Leukemic cells also accumulate rapidly in the spleen (FIG. 1G), followed by the blood (FIG. 1H). This sequence suggests initial engraftment of leukemic cells in the marrow and spleen followed by migration into the bloodstream.

### Leukemia Decreases Osteoblastic Cells

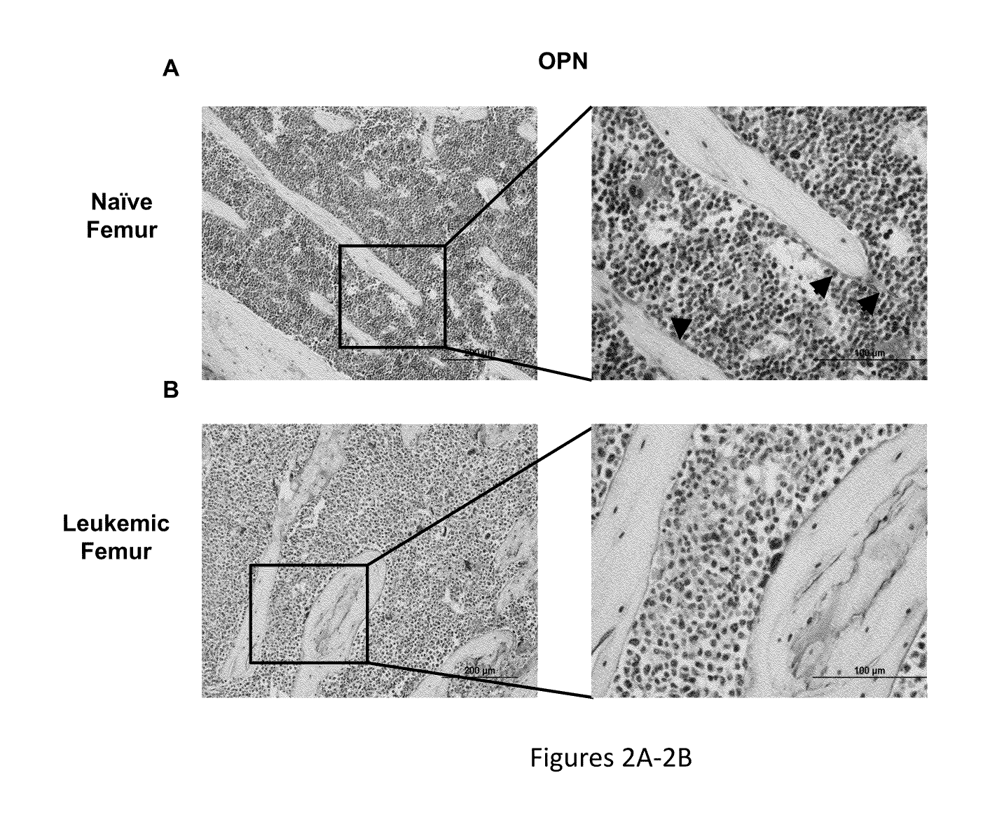

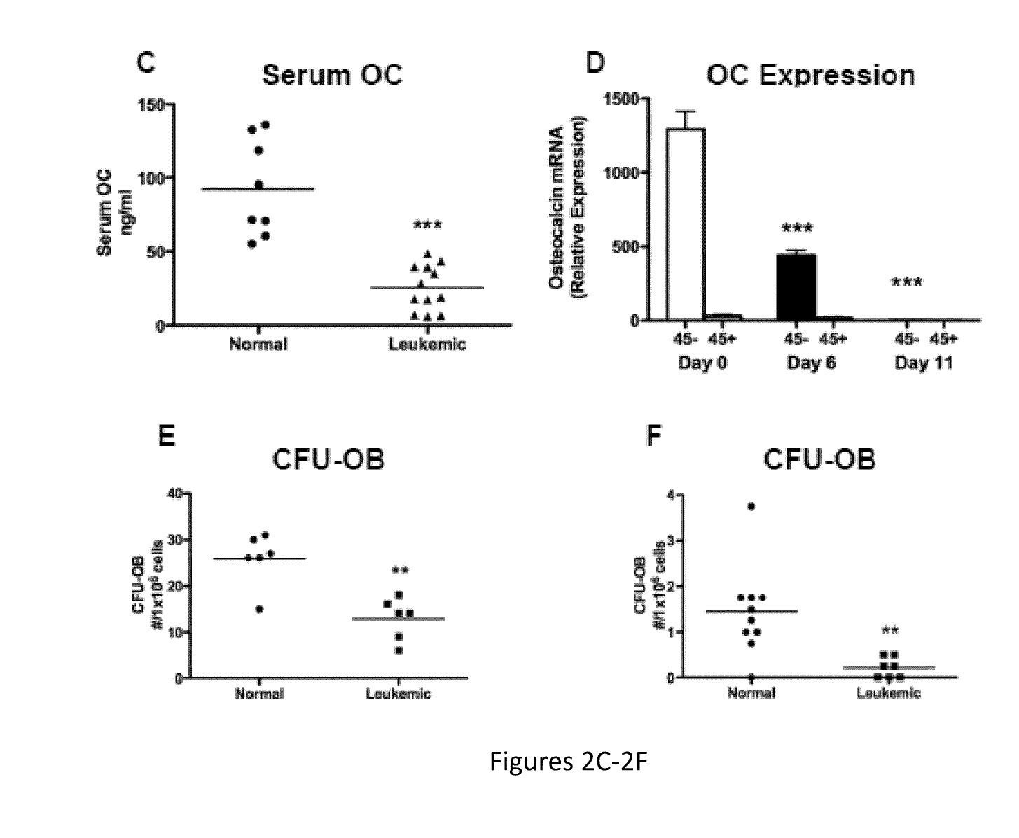

Examination by immunohistochemistry of sections from the long bones demonstrated a severe decrease in osteopontin+ endosteal osteoblastic cells in leukemic compared to normal mice (FIG. 2A-2B). Osteoblastic cells actively lay down bone; therefore a decrease in bone formation would be expected with osteoblastic inhibition. Global bone formation correlates well with serum osteocalcin levels, which were strongly suppressed in leukemic mice compared to sex and age-matched controls (FIG. 2C). Since osteocalcin is specifically expressed in mature osteoblastic cells, osteocalcin expression was measured in cells obtained from the long bones (femur and tibia) by collagenase digestion after bulk hematopoietic cells are removed by flushing. Osteocalcin expression was present only in non-hematopoietic, CD45− cells and in leukemic mice there was a nearly 400-fold decrease in osteocalcin expression (FIG. 2D), demonstrating a loss of osteocalcin+ cells from the marrow microenvironment. Osteocalcin expression was already decreased at day 6, when only 10-15% of marrow mononuclear cells are leukemic (FIG. 1F). To quantify the presence of osteoprogenitors in this cell pool, bone nodule analysis was performed and it was found that cells from the leukemic mice had reduced capacity to form alkaline phosphatase and von kossa positive colonies (FIG. 2E). Notably, a small number of bone-attached CD45+ cells were still present, and in the cultures from leukemic mice, leukemic cells were detected by fluorescence, although they represented only a very small proportion of the total cells. To identify more primitive osteoprogenitors and exclude leukemic cells from the bone nodule cultures from leukemic mice, marrow from leukemic mice was depleted of leukemic cells by FACS prior to plating. Marrow from leukemic mice had reduced osteoprogenitor numbers compared to normal sorted marrow (FIG. 2F). These data suggest that, in addition to impaired function, osteoblastic and to a lesser degree osteoprogenitor numbers are decreased in the long bones of leukemic mice.

### Leukemia Induces Bone Loss

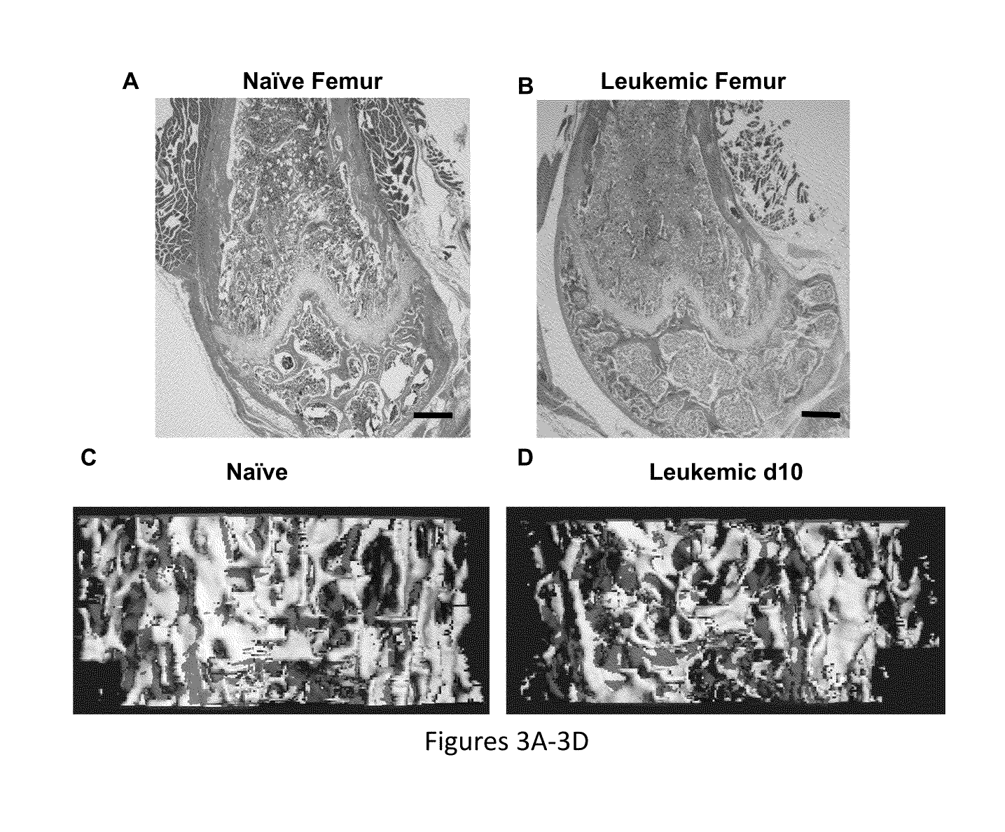

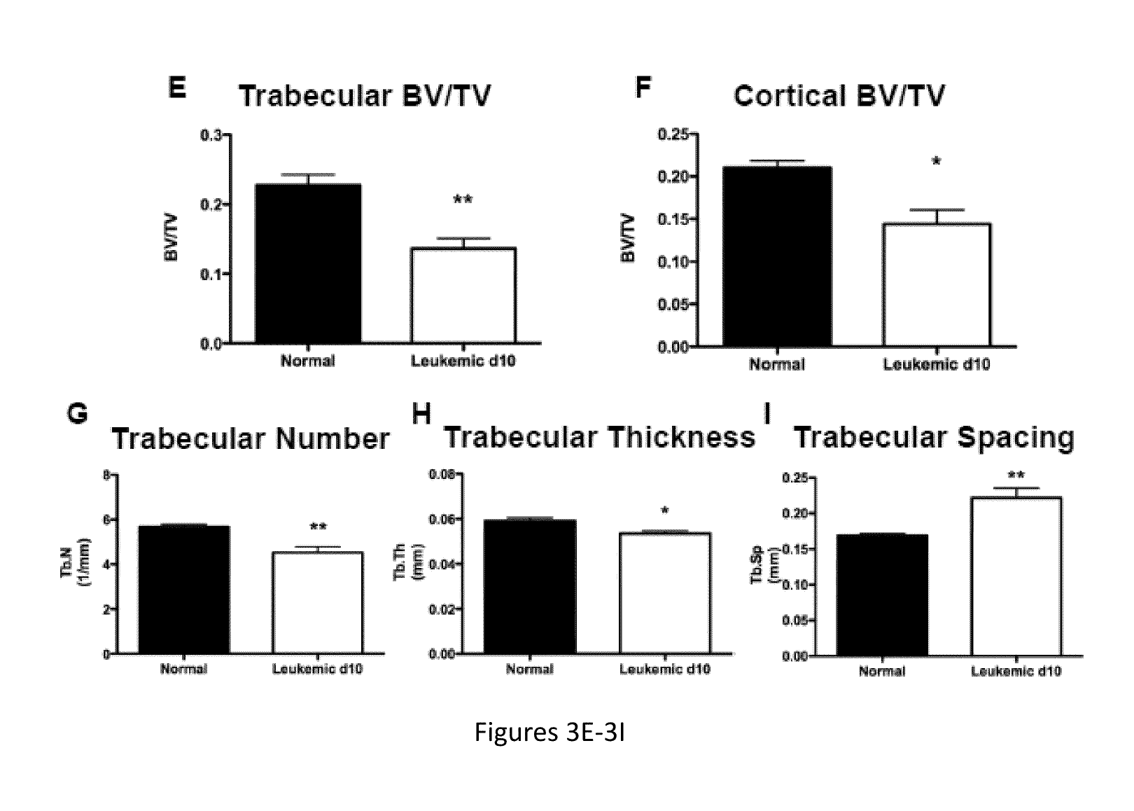

Functional inhibition of osteoblastic cells could result in a decrease in bone structures. Leukemic mice exhibited qualitative loss of trabecular structures, both in the metaphyseal region and the secondary ossification centers of the long bones, as well as cortical thinning (FIG. 3A-3B). At day 10, quantification of mineralized bone volume (BV/TV) by micro-CT analysis demonstrated severe loss of trabecular (FIG. 3C-3E) and cortical (FIG. 3F) bone in femora and tibiae of leukemic mice compared to age and sex-matched controls. Trabecular number and thickness were decreased (FIG. 3G-3H) and trabecular spacing was increased (FIG. 3I) in leukemic mice, consistent with alterations in the bone microenvironment and loss of trabecular bone volume. Therefore, in mice, development of AML results in net loss of mineralized bone.

### Leukemic Environment Transiently Increases Osteoclast Numbers In Vivo

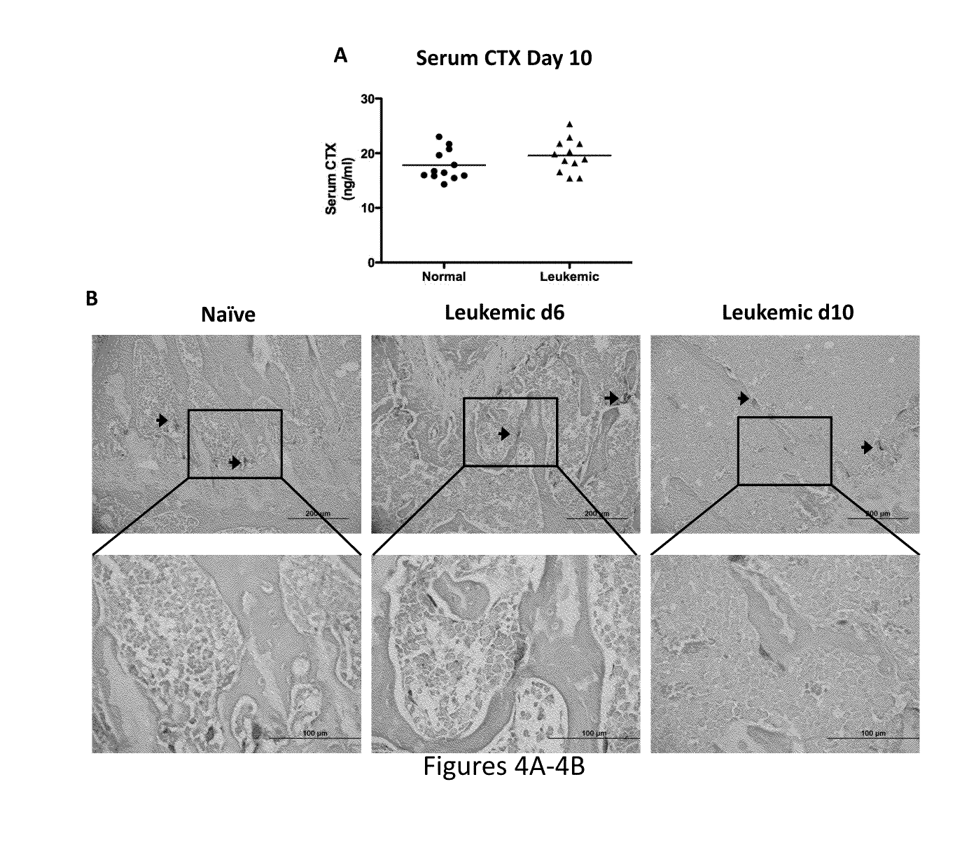

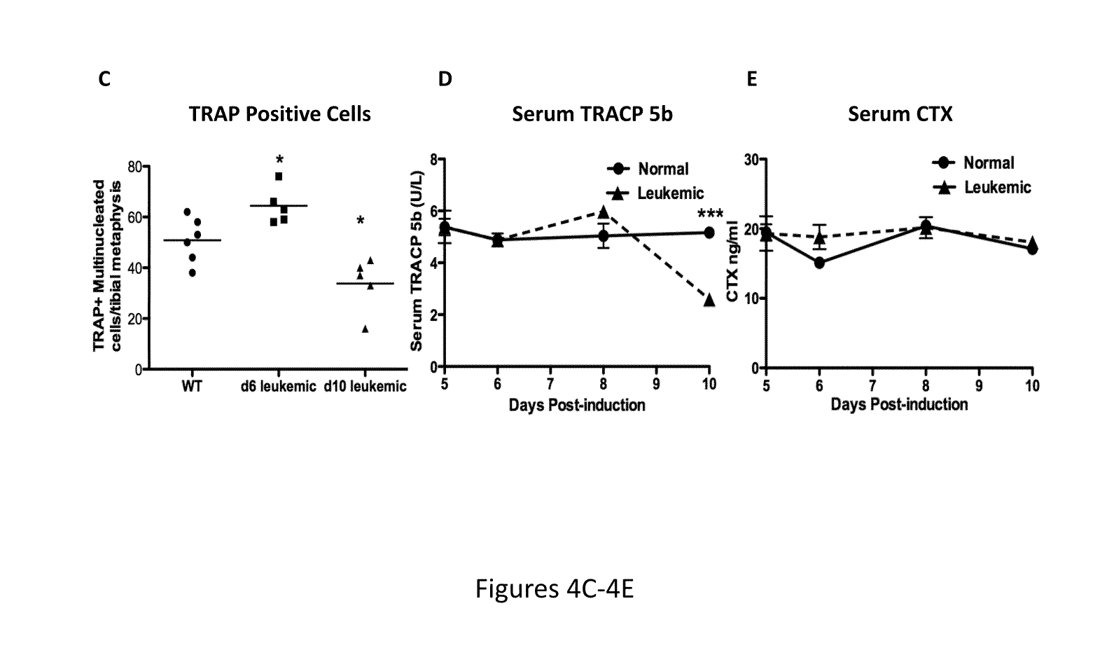

Bone formation and resorption are closely linked; therefore bone loss could result from increased bone resorption, in addition to decreased bone formation. C-terminal telopeptides (CTX) are released from the bone matrix when it is resorbed and are an established measure of global bone resorption (1993, Risteli et al., Clin Chem. 39:635-640). When serum CTX levels were measured in the sera from leukemic and normal mice, there was no measured change in CTX levels from leukemic mice at any time during disease progression (FIG. 4A).

To quantify osteoclasts, histological sections from normal and leukemic mice were stained for Tartrate Resistant Acid Phosphatase (TRAP) activity. Multinucleated TRAP+ cells were scored as osteoclasts (FIG. 4B). In leukemic mice, osteoclasts were mildly increased 6 days after induction of disease, but were decreased by 10 days in leukemic mice compared to controls (FIG. 4B-4C). Measurement of serum TRACP 5b has been validated as a measure of global osteoclast numbers (2005, Aya et al., J Clin Invest. 115:1848-1854). In normal mice, the TRACP 5b serum levels were stable, contrasting with leukemic mice in which global TRACP 5b levels were initially mildly increased and fell below normal by day 10 mirroring the histologic findings (FIG. 4D). These data suggest that, in the model tested herein, there is an initial and transient increase in osteoclastic cells, but as the disease progresses to overt leukemia, osteoclastic cell numbers decline.

Leukemic Cells do not Differentiate into Osteoclasts and do not Resorb Bone Matrix

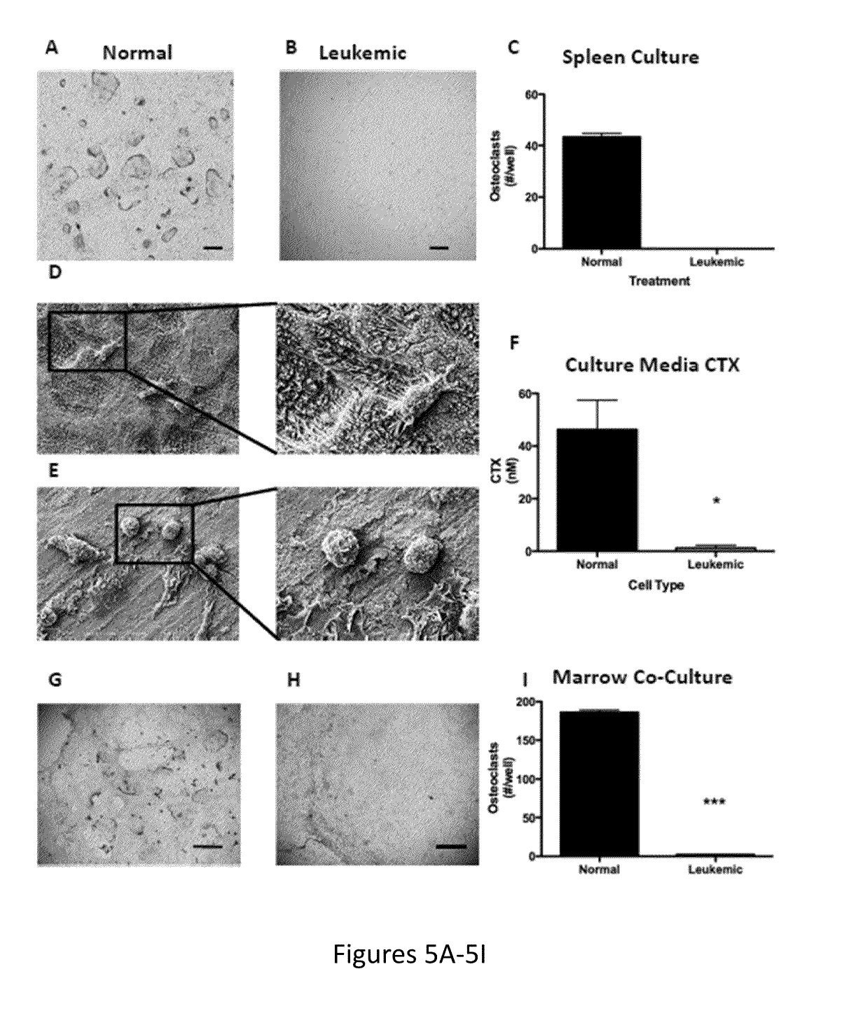

As osteoclasts are derived from the myeloid lineage, it is possible that leukemic cells may differentiate into osteoclasts. Therefore, the ability of spleen-derived leukemic cells to differentiate into osteoclasts in vitro was evaluated. Under osteoclastogenic conditions, cells from normal spleens produce abundant osteoclasts (FIG. 5A-5C). In striking contrast, GFP+/YFP+ leukemic cells isolated from spleens produce no TRAP+ osteoclasts (FIG. 5B-5C).

Although leukemic cells do not differentiate into TRAP+ osteoclasts in vitro, they may be able to resorb bone matrix. To determine if leukemic cells resorb bone directly, GFP+/YFP+ cells from the spleens of leukemic mice were sorted and cultured on bovine bone wafers. Scanning electron microscopic images were obtained to visualize the surface of the bone wafers, where osteoclastic activity is identified by the presence of resorption pits with a rough appearance that were abundant in normal controls (FIG. 5D). While viable leukemic cells were observed to adhere to the surface of the bone wafers, the bone matrix in wells containing leukemic cells remained smooth and lacked resorption pits (FIG. 5E). The amount of bone resorption was determined by the quantification of bovine CTX in the culture media. Normal osteoclast precursors under osteoclastogenic conditions readily resorb bone, as was demonstrated by the increase in bovine CTX. In contrast, leukemic cells under the same conditions did not increase bovine CTX (FIG. 5F). These data suggest that GFP+/YFP+ leukemic cells do not directly contribute to bone resorption.

To determine if the initial increase in osteoclasts in vivo was the result of pro-osteoclastogenic signals from the leukemic clone, marrow cells from either leukemic or normal mice were cocultured with a normal osteoblastic feeder layer in the presence of 1.25 dihydroxyvitamin D3. Under these culturing conditions, osteoclast formation was much more abundant in normal marrow cells as compared to leukemic (FIG. 5G-5I). These data confirm the inability of leukemia cells to form mature osteoclasts in vitro and suggest that leukemic cells require a full leukemic microenvironment to stimulate the in vivo transient increase in osteoclasts.

Treatment with Zoledronic Acid Blocks Bone Resorption but Only Partially Rescues Bone Loss in Leukemic Mice

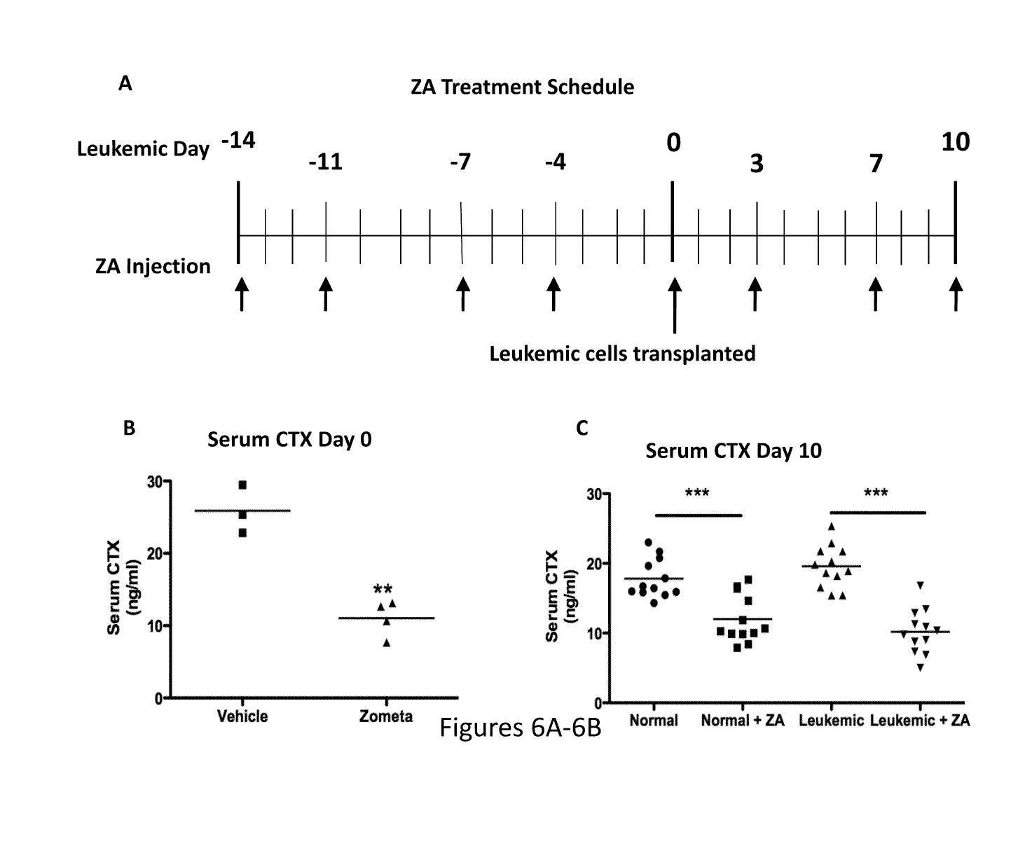

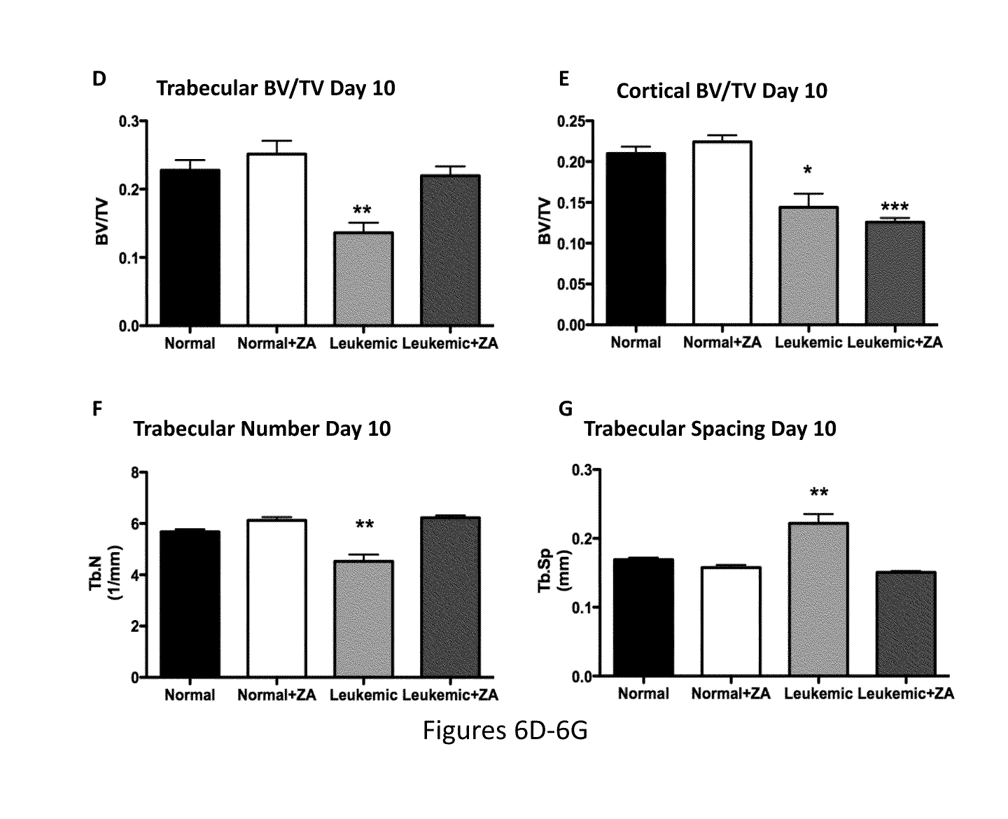

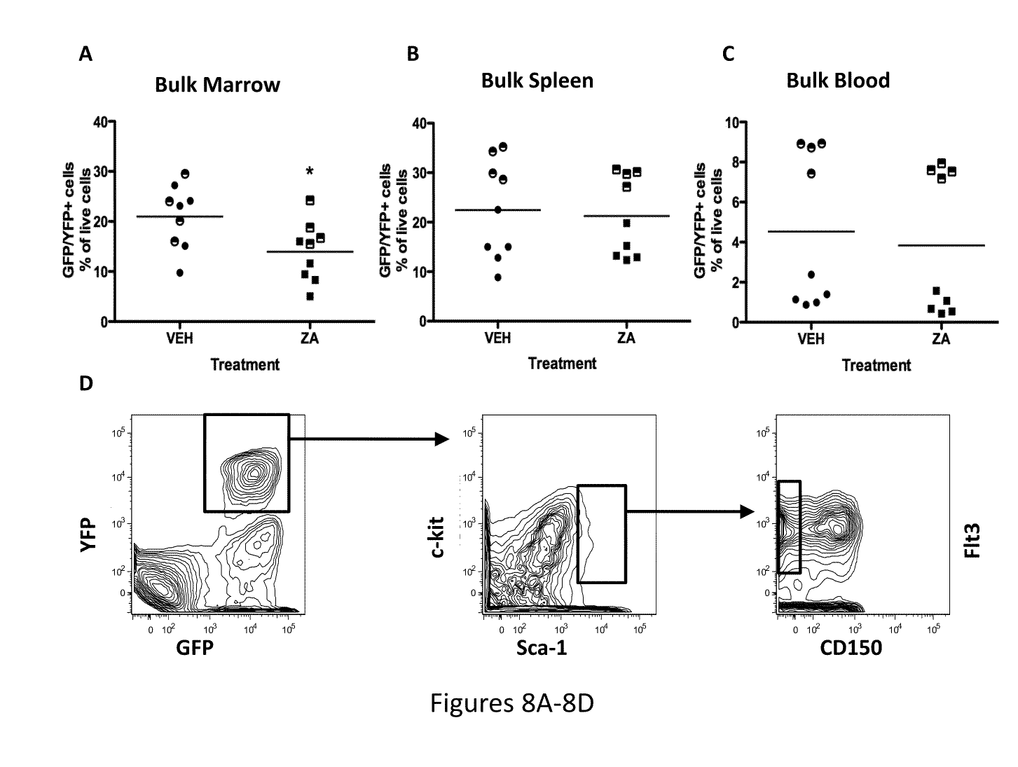

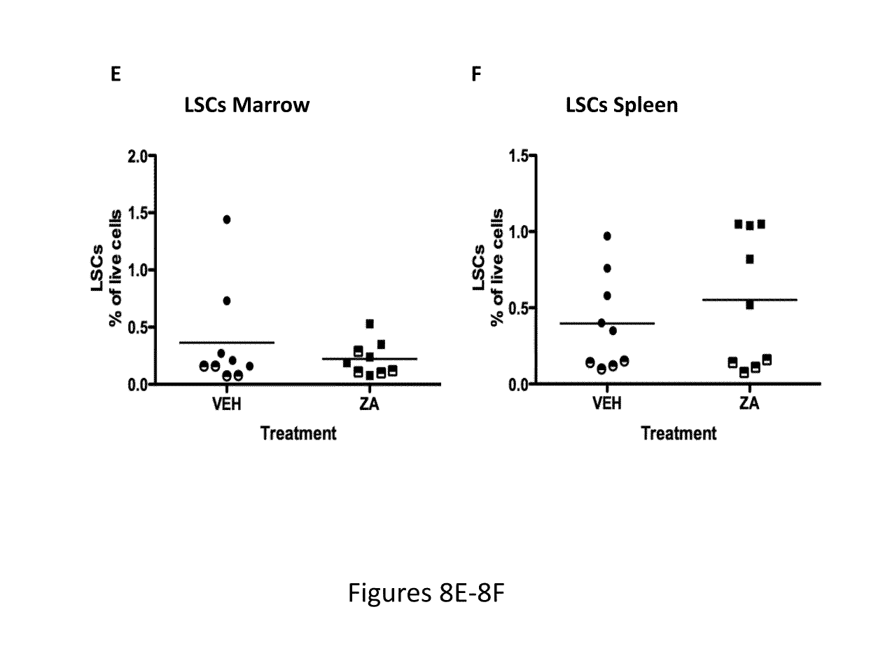

To determine whether loss of bone formation or increased numbers of osteoclasts was responsible for the loss of bone in leukemic animals, mice were treated with Zoledronic acid (ZA), a highly potent osteoclastic inhibitor (1994, Green et al., J Bone Miner Res. 9:745-751) prior to transplantation with leukemic cells (FIG. 6A). This treatment was sufficient to inhibit the activity of osteoclasts (FIG. 6B). The serum levels of osteocalcin, a well-established bone formation marker, were significantly decreased in normal ZA-treated mice (142±28 vs 49±1 ng/ml, vehicle vs. ZA-treated mice p=0.0296). This homeostatic compensatory decrease in bone formation in response to effective osteoclastic inhibition explains the lack of significant ZA-dependent increases in bone volumes in wildtype mice (FIG. 6D-6E). In leukemic mice treated with ZA serum CTX dropped to subnormal levels (FIG. 6C), confirming the inhibitory effect of ZA on osteoclasts in the setting of leukemia. Loss of trabecular bone volume was ameliorated by treatment with ZA (FIG. 6D). Trabecular number (FIG. 6F) and spacing (FIG. 6G) were also rescued, consistent with ZA-dependent protection of the trabecular bone volume loss induced by leukemia. In contrast, cortical bone volume loss remained unchanged by inhibition of resorption (FIG. 6E). When disease burden was quantified in leukemic mice treated with ZA, there was a mild decrease in disease burden in the marrow (FIG. 8A), but not in spleen (FIG. 8B) or peripheral blood (FIG. 8C). In this model the phenotype of the leukemia stem cell (LSC) has been previously defined (2007, Neering et al., Blood 110:2578-2585) (FIG. 8D), however treatment with ZA did not result in measurable changes in the frequency of LSC in marrow (FIG. 8E) or spleen (FIG. 8F). Moreover, there were no changes in progression of disease or mortality in ZA-treated leukemic mice (data not shown). These data suggest that inhibition of bone resorption only partially reverses leukemic-induced bone loss without significantly changing disease progression.

### Expression of the Chemokine CCL3 is Increased in Malignant Marrow Cells in Leukemic Mice

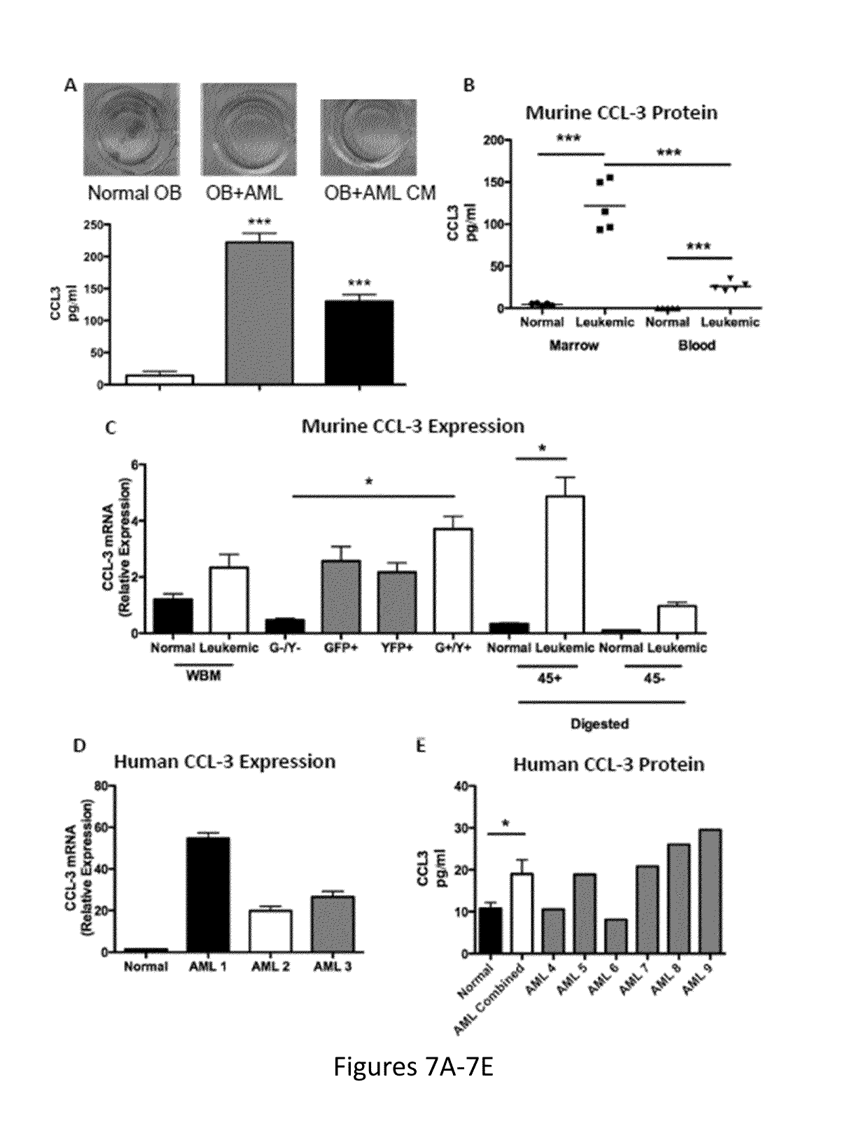

Osteoblastic inhibition by leukemia may be mediated by cell contact and/or by secreted molecules. CFU-OB co-cultures of wildtype pre-osteoblasts with murine leukemic cells or with conditioned media generated from murine leukemic cells both showed a similar lack of alkaline phosphatase positive colonies (FIG. 7A) suggesting that a secreted factor(s) is (are) sufficient to suppress colony formation. CCL3 is a pro-inflammatory cytokine in the CC chemokine family that has been implicated in the pro-osteoclastic actions observed in multiple myeloma (2001, Han et al., Blood 97:3349-3353; 2003, 2003, Lentzsch et al., Blood 101:3568-3573). In addition, data have recently suggested that CCL3 inhibits osteoblastic cells in mice and in human in vitro studies (2011, Vallet et al., Leukemia). The level of CCL3 in the media was elevated in both co-cultures and conditioned media (FIG. 7A). CCL3 protein levels were also increased in the marrow plasma and blood serum from leukemic animals compared to normal controls (FIG. 7B). To determine the cellular source of CCL3, mRNA was measured by quantitative real time RT-PCR in total mRNA of marrow mononuclear cells, cells digested from bone fragments of the hindlimbs that were separated based on surface CD45 positivity, and sorted GFP+/YFP+ leukemic cells. Bone-associated CD45+ cells from leukemic mice expressed higher levels of CCL3 mRNA compared to normal controls (FIG. 7C). In sorted cells, GFP+/YFP+ leukemic cells demonstrated the highest level of CCL3 expression (FIG. 7C). To assess whether CCL3 is also upregulated in patients with AML, CCL3 expression was quantified in sorted CD34+CD38−CD123+ human marrow cells, and found to be increased in marrow samples from AML patients compared to normal controls (FIG. 7D). In addition CCL3 protein levels were elevated in the marrow plasma from AML patients compared to normal controls (FIG. 7F). This data is in agreement with the analysis of microarray data previously published by Bullinger et al., in which 58 of 75 AML samples had elevated levels of CCL3 (2004, N Engl J. Med. 350:1605-1616).

### Example 2

### Characterization of CCL Knockout Cells and Mice

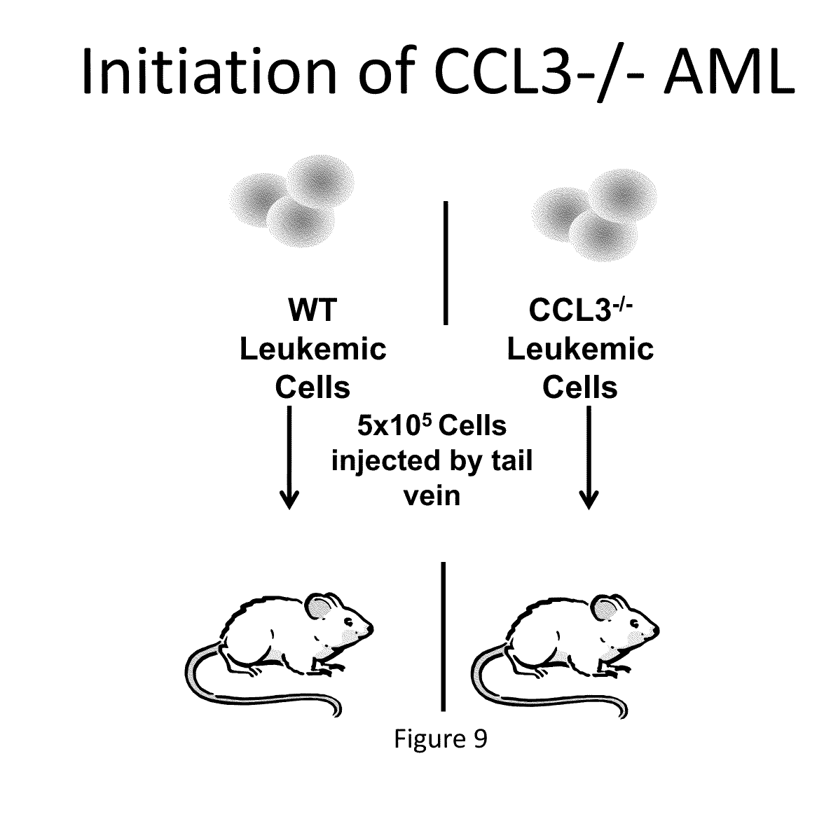

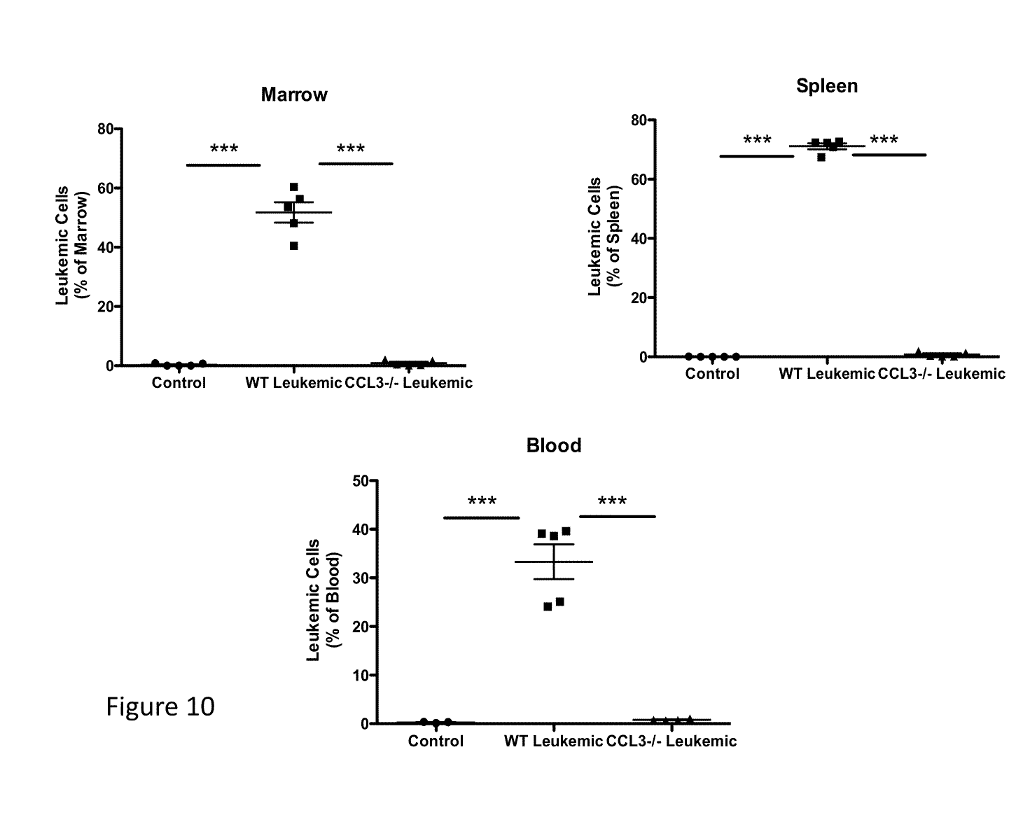

Experiments were conducted to assess the effects of inhibiting CCL in vivo. For example, a complete lack of leukemia progression was observed when leukemia cells are CCL3 knockouts. Briefly, either wild-type or CCL^{-/-} leukemic cells were injected into a mouse (FIG. 9). CCL3^{-/-} AML cells failed to initiate leukemia in WT recipient mice (FIG. 10). The results from this experiment demonstrate that CCL3 expression is related to progression of leukemia whereby inhibiting CCL3 helps to prevent and treat leukemia. Accordingly, the results support methods of treating cancer (e.g., leukemia) by inhibiting CCL3 or CCL3 signaling.

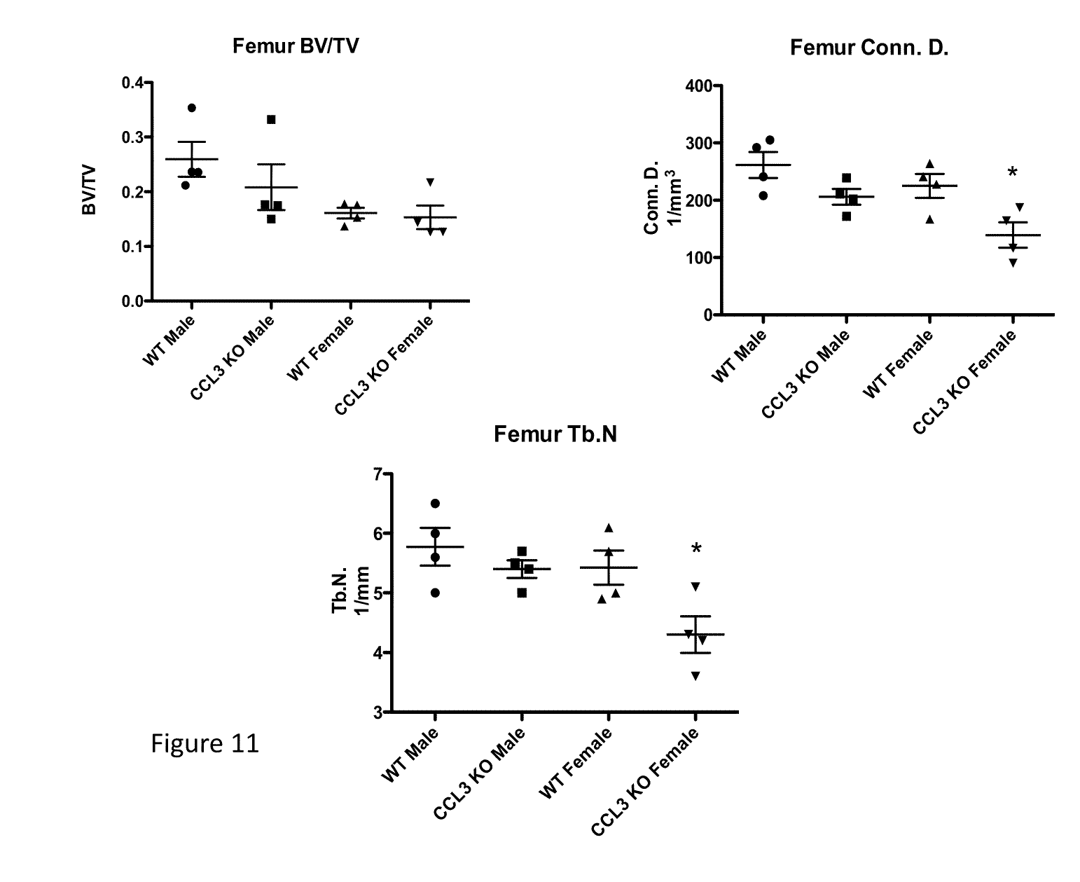

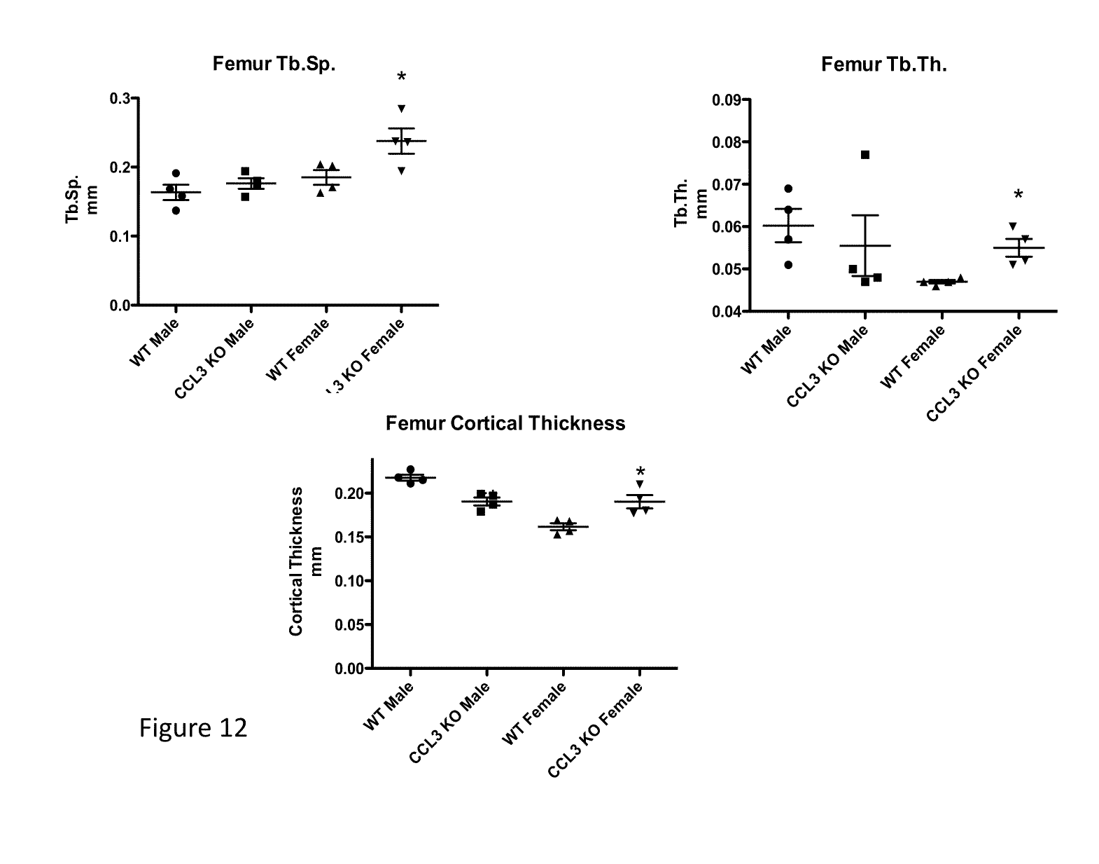

The next experiment was performed to assess bone phenotype from knocking out CCL3. The results demonstrate that there is a bone phenotype associated with knocking out CCL3. The results demonstrate that leukemia produced CCL3 is altering the bone microenvironment to favor malignant hematopoiesis over benign (FIGS. 11 and 12). Moreover, FIG. 12 shows an increase in cortical thickness in CCL3 knockout mice which suggests that CCL3 inhibition may be a strategy for treatment of bone loss.

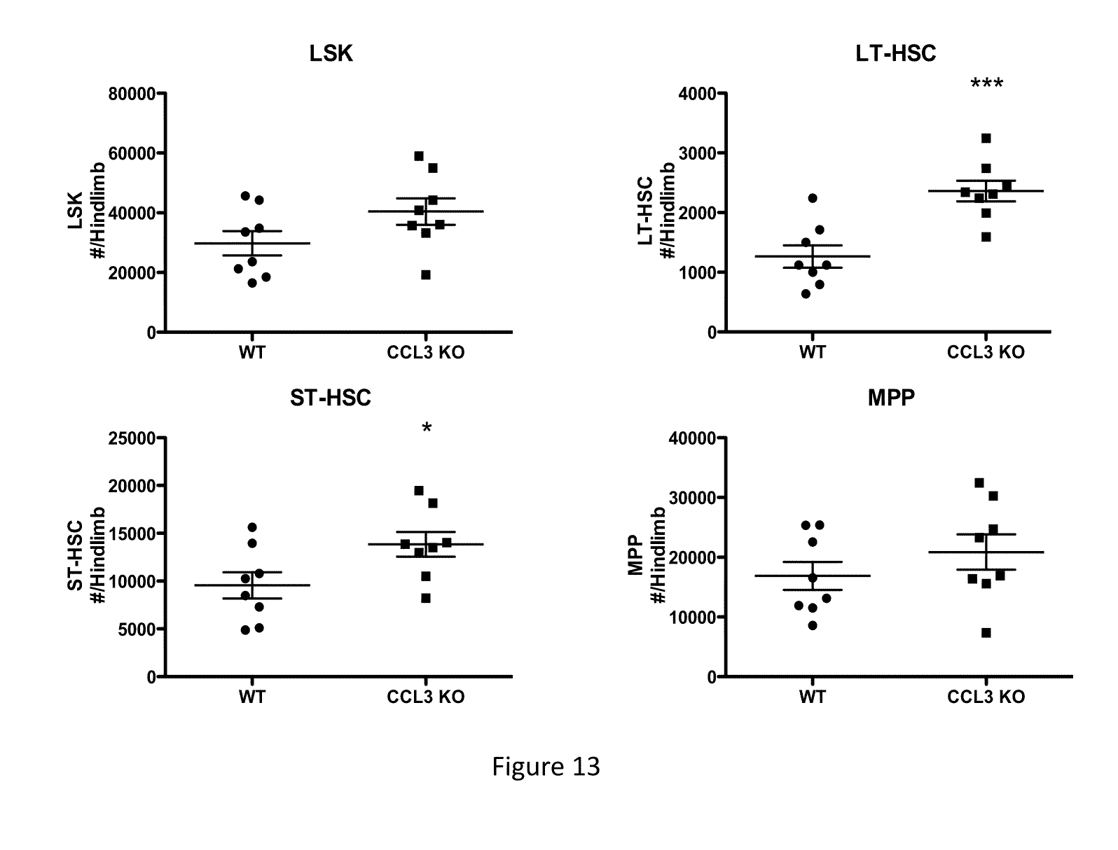

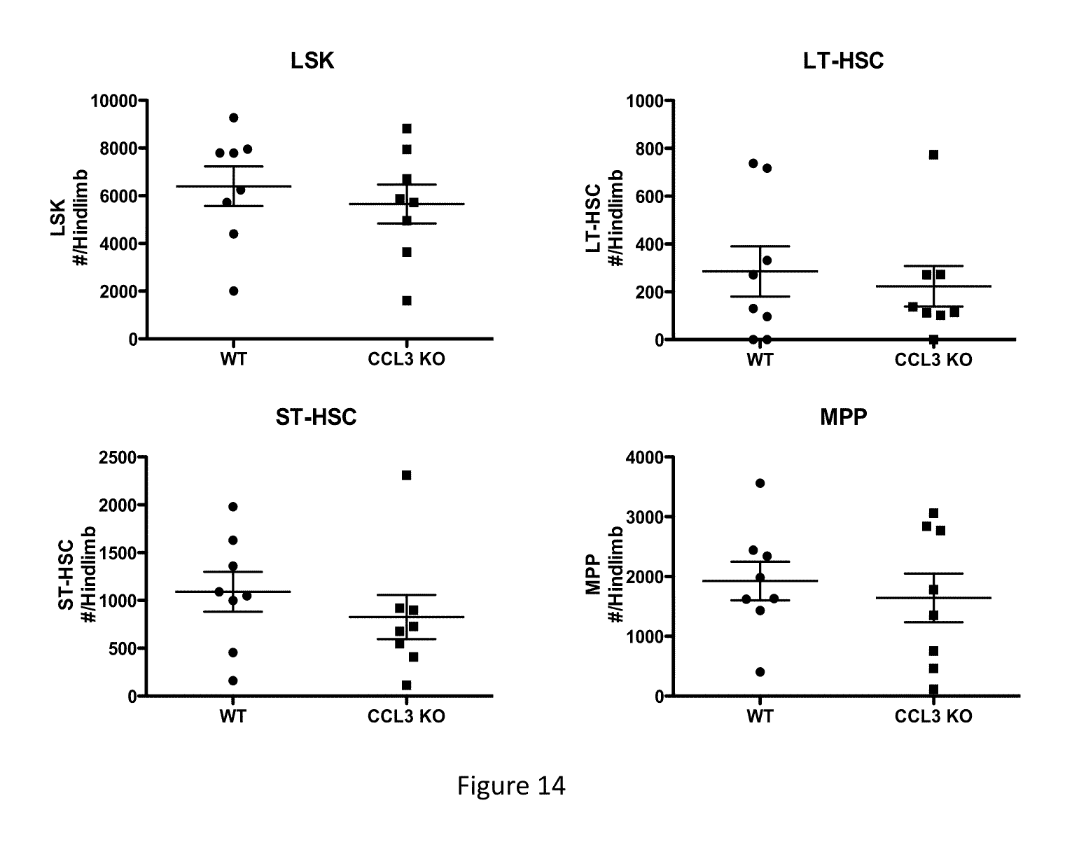

The next experiment was performed to assess hematopoietic stem cells from CCL3 knockout mice. FIGS. 13 and 14 demonstrate that in non-leukemic CCL3 knockout mice, there is no deficit in hematopoietic stem cell numbers in the marrow, or spleen, suggesting that any strategy to inhibit CCL3 signaling in the context of leukemia would have no detrimental effects on the normal hematopoietic stem cell populations. Surprisingly non-leukemic CCL3 knockout mice actually showed a 2 fold increase in LT-HSCs in the bone marrow. This suggests that inhibition of CCL3 signaling could be used as a strategy for expansion of HSCs.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating a pathology associated with myeloid neoplasm in a subject, said method comprising administering a therapeutically effective amount of a CCL3 inhibitor or a CCL3 receptor inhibitor to the subject, wherein the subject has myeloid neoplasm, and wherein after the CCL3 inhibitor or CCL3 receptor inhibitor is administered to the subject, the pathology is prevented.

2. The method of claim 1, wherein the CCL3 inhibitor is at least one selected from the group consisting of: a chemical compound, a protein, a peptide, a peptidomemetic, an antibody, a ribozyme, a small molecule chemical compound, and an antisense nucleic acid molecule,
and wherein the CCL3 receptor inhibitor is one selected from the group consisting of: a chemical compound, a protein, a peptide, a peptidomemetic, an antibody, a ribozyme, a small molecule chemical compound, and an antisense nucleic acid molecule.

3. The method of claim 1, wherein the myeloid neoplasm is at least one selected from the group consisting of: a myeloproliferative disorder, acute myeloid leukemia (AML), chronic myeloid leukemia (CML), chronic myelomonocytic leukemia (CMML) and myelodysplastic syndrome (MDS).

4. The method of claim 1, wherein the pathology that is treated is at least one selected from the group consisting of: osteoclast/osteoblast imbalance, inhibition of osteoblast function, bone loss, disregulation of the hematopoietic stem cell microenvironment, abnormal hematopoietic recovery, pancytopenia, anemia, thrombocytopenia, neutropenia, bicytopenia, and erythrocytopenia.

5. The method of claim 1, wherein the subject is a human.

6. The method of claim 1, wherein the CCL3 receptor is at least one selected from the group consisting of CCR1 and CCR5.

7. A method of preventing a pathology associated with myeloid neoplasm in a subject, said method comprising administering a therapeutically effective amount of a CCL3 inhibitor or a CCL3 receptor inhibitor to the subject, wherein the subject has myeloid neoplasm, and wherein after the CCL3 inhibitor or CCL3 receptor inhibitor is administered to the subject, the pathology is prevented.

8. The method of claim 7, wherein the CCL3 inhibitor is at least one selected from the group consisting of: a chemical compound, a protein, a peptide, a peptidomemetic, an antibody, a ribozyme, a small molecule chemical compound, and an antisense nucleic acid molecule,
and wherein the CCL3 receptor inhibitor is one selected from the group consisting of: a chemical compound, a protein, a peptide, a peptidomemetic, an antibody, a ribozyme, a small molecule chemical compound, and an antisense nucleic acid molecule.

9. The method of claim 7, wherein the myeloid neoplasm is at least one selected from the group consisting of: a myeloproliferative disorder, acute myeloid leukemia (AML), chronic myeloid leukemia (CML), chronic myelomonocytic leukemia (CMML) and myelodysplastic syndrome (MDS).

10. The method of claim 7, wherein the pathology that is treated is at least one selected from the group consisting of: osteoclast/osteoblast imbalance, inhibition of osteoblast function, bone loss, disregulation of the hematopoietic stem cell microenvironment, abnormal hematopoietic recovery, pancytopenia, anemia, thrombocytopenia, neutropenia, bicytopenia, and erythrocytopenia.

11. The method of claim 7, wherein the subject is a human.

12. The method of claim 7, wherein the CCL3 receptor is at least one selected from the group consisting of CCR1 and CCR5.

13. A composition comprising a CCL3 inhibitor or a CCL3 receptor inhibitor, wherein the CCL3 inhibitor is one selected from the group consisting of: a chemical compound, a protein, a peptide, a peptidomemetic, an antibody, a ribozyme, a small molecule chemical compound, and an antisense nucleic acid molecule,
and wherein the CCL3 receptor inhibitor is one selected from the group consisting of: a chemical compound, a protein, a peptide, a peptidomemetic, an antibody, a ribozyme, a small molecule chemical compound, and an antisense nucleic acid molecule.

14. The composition of claim 13, wherein the CCL3 receptor is at least one selected from the group consisting of CCR1 and CCR5.

15. A method of expanding hematopoietic stem cells (HSCs), said method comprising contacting HSCs with an inhibitor of CCL3 or a CCL3 receptor inhibitor.

16. The method of claim 15, wherein the CCL3 inhibitor is at least one selected from the group consisting of: a chemical compound, a protein, a peptide, a peptidomemetic, an antibody, a ribozyme, a small molecule chemical compound, and an antisense nucleic acid molecule,
and wherein the CCL3 receptor inhibitor is one selected from the group consisting of: a chemical compound, a protein, a peptide, a peptidomemetic, an antibody, a ribozyme, a small molecule chemical compound, and an antisense nucleic acid molecule.

17. The method of claim 16 wherein the CCL3 receptor is at least one selected from the group consisting of CCR1 and CCR5.

18. A method of treating bone loss in a subject, said method comprising administering a therapeutically effective amount of a CCL3 inhibitor or a CCL3 receptor inhibitor to a subject in need thereof.

19. The method of claim 17, wherein the CCL3 inhibitor is at least one selected from the group consisting of: a chemical compound, a protein, a peptide, a peptidomemetic, an antibody, a ribozyme, a small molecule chemical compound, and an antisense nucleic acid molecule,
and wherein the CCL3 receptor inhibitor is one selected from the group consisting of: a chemical compound, a protein, a peptide, a peptidomemetic, an antibody, a ribozyme, a small molecule chemical compound, and an antisense nucleic acid molecule.

20. The method of claim 18 wherein the CCL3 receptor is at least one selected from the group consisting of CCR1 and CCR5.


SEQUENCE LISTING (6 sequences)

SEQ ID NO: 1 - DNA, 19 bp, Mus musculus
gccactgccgcatcctctt

SEQ ID NO: 2 - DNA, 21 bp, Mus musculus
ggaaccgctcgttgccaatag

SEQ ID NO: 3 - DNA, 21 bp, Mus musculus
ccgcctacaaacgcatctacg

SEQ ID NO: 4 - DNA, 23 bp, Mus musculus
gagagaggacagggaggatcaag

SEQ ID NO: 5 - DNA, 23 bp, Mus musculus
aaggatacaagcagcagcgagta

SEQ ID NO: 6 - DNA, 24 bp, Mus musculus
tgcagagtgtcatggtacagagaa